(19) United States
(12) Patent Application Publication
(10) Pub. No.: US 2006/0058617 A1
(43) Pub. Date: Mar. 16, 2006

(54) Method and system for displaying medical images

(76) Inventors: Daisuke Sano, Tokyo (JP); Takefumi Uesugi, Tokyo (JP); Atsuhiko Kasahi, Yokohama (JP); Kenji Noda, Tokyo (JP)

Correspondence Address: OSTROLENK FABER GERB & SOFFEN, 1180 AVENUE OF THE AMERICAS, NEW YORK, NY, 100368403, US

(21) Appl. No.: 11/200,945

(22) Filed: Aug. 10, 2005

(30) Foreign Application Priority Data
Aug. 24, 2004 (JP) 2004-244229

(51) Int. Cl. A61B 5/05

(52) U.S. Cl. 600/407

(57) ABSTRACT

In a medical image display system, a gas supply unit is configured to supply first gas into a first cavity and second gas into a second cavity. A switching display unit is connected to a display and is configured to determine whether the first gas or the second gas is supplied from the gas supply unit. The switching display unit is configured to switchably display the first medical image and the second medical image on the screen of the display based on the determined result.

### CROSS REFERENCE TO RELATED APPLICATION

This application is based upon the prior Japanese Patent Application 2004-244229 filed on Aug. 24, 2004 and claims the benefit of priority therefrom so that the descriptions of which are all incorporated herein by reference.

### BACKGROUND OF THE INVENTION

1. Field of the Invention

The present invention relates to a method and a system for displaying medical images of a body.

2. Description of the Related Art

In recent years, laparoscopic surgeries have been practiced extensively. The laparoscopic surgery is executed for treating a patient with minimally invasive capability.

Specifically, in the laparoscopic surgeries, for example, a first trocar for introducing a rigid endoscope, referred to as “rigidscope”, for observation to a body cavity of a patient is inserted thereinto. In addition, a second trocar for introducing a treatment tool to a site to be treated is inserted thereinto.

In such a laparoscopic surgery, an insufflator has been used for supplying carbon dioxide gas (hereinafter also referred to as CO₂) as insufflation gas into an abdominal cavity of the patient to ensure the rigidscope field and a space to manipulate the treatment tool.

Conventionally, some types of insufflators each for supplying carbon dioxide gas into one of body cavities, such as an abdominal cavity of the patient, have been prepared.

For example, Japanese Unexamined Patent Publication No. 2000-139830 discloses a gas supplying apparatus designed to feed a control signal to a pressure-regulating valve when gas flow volume does not reach a predetermined value. The control signal causes the pressure-regulating valve to increase the pressure of the output gas to control the amount thereof, thereby keeping an internal pressure of a living body at the predetermined value.

Moreover, Japanese Unexamined Patent Publication No. 8-256972 discloses an insufflator having a plurality of electro magnetic valves for controlling a state of gas flowing through a gas delivery channel extending from a gas supply source to an insufflation tool. Specifically, the insufflator is designed so that the plurality of electro magnetic values is integrated with a manifold valve, allowing the gas-flow state controlling section to become compact.

Furthermore, Japanese Unexamined Patent Publication No. 2000-139823 discloses an insufflation system for insufflating air into a lumen to keep constant the pressure inside of the lumen.

In the meanwhile, when diagnosing and treating a lumen, such as the stomach, the large intestine, or the like of a patient as one of the body cavities thereof, a flexible endoscope, referred to as “flexiblescope”, and a treatment tool therefor have been used. The flexiblescope has one thin and flexible end portion to be used as an access site into the lumen. The treatment tool for the flexiblescope is designed so that its forceps channel is inserted into the flexiblescope to project through an opening formed in the head of the one end portion of the flexiblescope.

When executing curative intervention, such as diagnosis and treatment of a lumen, such as the stomach, the large intestine or the like of a patient under such monitored conditions with the flexiblescope, in some cases, gas for lumens is injected into the lumen. The injection of gas aims at securing the flexiblescope field and a space to manipulate the treatment tool.

In these cases, the gas to be supplied into the lumen can be transferred with a gas supply pump. As the gas for lumens, air has been generally applied, but the carbon dioxide gas can be used

Recently, as a new attempt, in the laparoscopic surgeries, the rigidscope is inserted into an abdominal cavity of a patient with the flexiblescope inserted into a lumen of the patient. This allows identification of a site to be treated in the patient based on an image of the inside of the abdominal cavity, which is obtained by the rigidscope, and that of the inside of the lumen, which is obtained by the flexiblescope.

Under such monitored conditions with both the rigidscope and flexiblescope, in some cases, for example, air as gas for lumens is injected through the flexiblescope into the lumen so that the lumen inflates.

When air is supplied into the lumen, it is difficult for the air to be absorbed into the living body. This may cause the lumen to remain inflated.

For this reason, when inserting the rigidscope into an abdominal cavity of a patient while inserting the flexiblescope into a lumen thereof, using an endoscope CO₂ regulator (hereinafter referred to as ECR) has been considered to supply carbon dioxide gas (CO₂), which is absorbed easily into the living body, into the lumen.

### SUMMARY OF THE INVENTION

The present invention has been made on the background.

According to one aspect of the present invention, there is provided a medical image display system including a display for displaying a first medical image related to a first body cavity and a second medical image related to a second body cavity on a screen. The medical image display system includes a gas supply unit configured to supply first gas into the first cavity and second gas into the second cavity, and a switching display unit connected to the display. The switching display unit is configured to determine whether the first gas or the second gas is supplied from the gas supply unit. The switching display unit is configured to switchably display the first medical image and the second medical image on the screen of the display based on the determined result.

According to another aspect of the present invention, there is provided a medical image display system including means for displaying a first medical image related to a first body cavity and a second medical image related to a second body cavity on a screen. The medical image display system includes means for supplying first gas into the first cavity and second gas into the second cavity, and means for determining whether the first gas or the second gas is supplied from the gas supply means. The supplying means is operative to switchably display the first medical image and the second medical image on the screen of the display based on the determined result.

According to a further aspect of the present invention, there is provided a method of displaying a first medical image related to a first body cavity and a second medical image related to a second body cavity on a screen. The method includes supplying first gas into the first cavity and second gas into the second cavity, determining whether the first gas or the second gas is supplied from the gas supply means, and switchably displaying the first medical image and the second medical image on the screen of the display based on the determined result.

### BRIEF DESCRIPTION OF THE DRAWINGS

Various aspects of the present invention will be more particularly described with reference to the accompanying drawings in which:

FIG. 1 is an overall structural view schematically illustrating the structure of a laparoscopic surgery system with a medical image display system according to a first embodiment of the present invention;

FIG. 2 is a view schematically illustrating a configuration example of an operation panel illustrated in FIG. 1;

FIG. 3 is a view schematically illustrating an example of a display panel illustrated in FIG. 1;

FIG. 4 is a view schematically illustrating a configuration example of a manually operable setting section and a display section provided on a front panel of the gas supply apparatus illustrated in FIG. 1;

FIG. 5 is a block diagram illustrating a schematic structure of the gas supply apparatus illustrated in FIG. 1;

FIG. 6 is a block diagram illustrating a schematic structure of an image processing unit of a system controller illustrated in FIG. 1;

FIG. 7 is a view illustrating an example of a composite image displayed on a screen of a monitor according to the first embodiment;

FIG. 8 is a flowchart schematically illustrating an example of operations of a control module illustrated in FIG. 6 according to the first embodiment of the present invention;

FIG. 9 is a flowchart schematically illustrating an example of operations of an image composition module illustrated in FIG. 6 according to the first embodiment of the present invention;

FIG. 10 is a view schematically illustrating an example of the composite image displayed on the screen of the monitor according to the first embodiment;

FIG. 11 is a view schematically illustrating another example of the composite image displayed on the screen of the monitor according to the first embodiment;

FIG. 12 is a view schematically illustrating a further example of the composite image displayed on the screen of the monitor according to the first embodiment;

FIG. 13 is a flowchart schematically illustrating another example of operations of the control module illustrated in FIG. 6 according to the first embodiment of the present invention;

FIG. 14 is a view schematically illustrating switching from a lumen image displayed on the screen of the monitor to an abdominal-cavity image according to the first embodiment;

FIG. 15 is a block diagram illustrating a functional structure of an image processing unit according to a second embodiment of the present invention;

FIG. 16 is an overall structural view schematically illustrating the structure of a surgical system according to a third embodiment of the present invention; and

FIG. 17 is a block diagram illustrating a functional structure of an image processing unit according to the third embodiment of the present invention.

### DETAILED DESCRIPTION OF EMBODIMENTS OF THE INVENTION

Embodiments of the present invention will be described hereinafter with reference to the accompanying drawings.

### First Embodiment

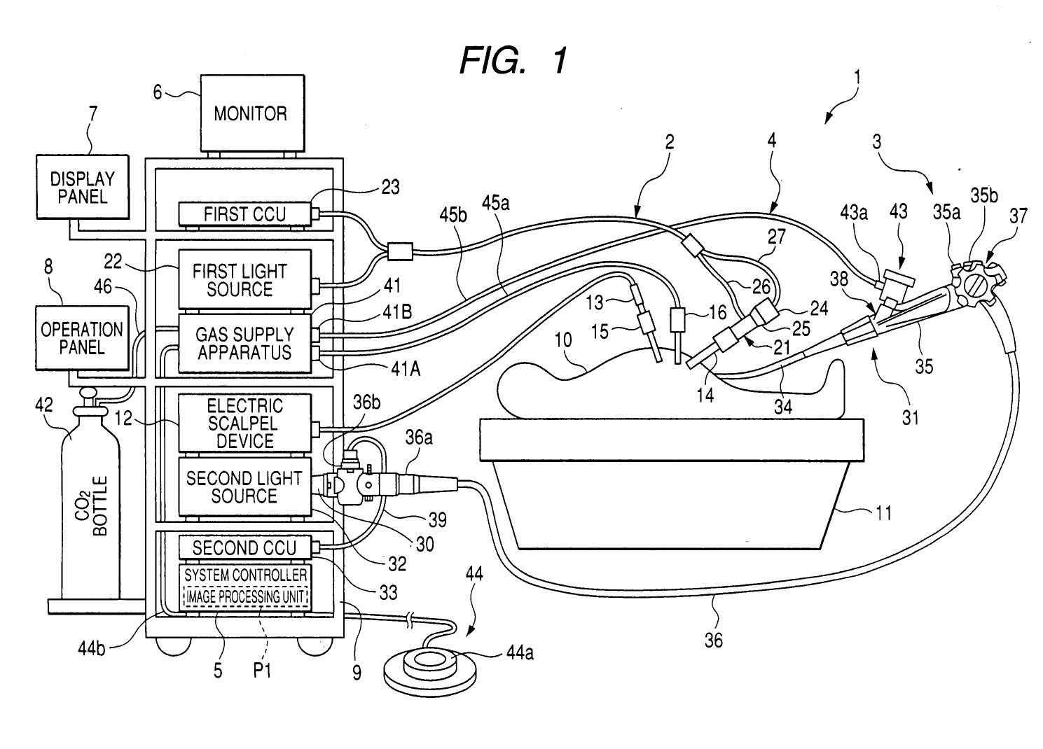

As shown in FIG. 1, a laparoscopic surgery system, referred to as a surgical system hereinafter, 1 with a medical image display system according to a first embodiment of the present invention has a first endoscope system 2, a second endoscope system 3, and a gas supply system 4.

The surgical system 1 has a system controller 5, a monitor 6 as a display device, a center display panel 7, a center operation panel 8, and a movable cart (trolley) 9.

Reference numeral 10 designates a patient (body), and reference numeral 11 designates an operation table that allows the patient 11 to lie thereon. Reference numeral 12 designates an electric scalpel device as an example of operation devices, which is mounted on the cart 9. The surgical system 1 has an electric scalpel 13 serving as an operation tool. The electric scalpel 13 is electrically connected to the electric scalpel device 12.

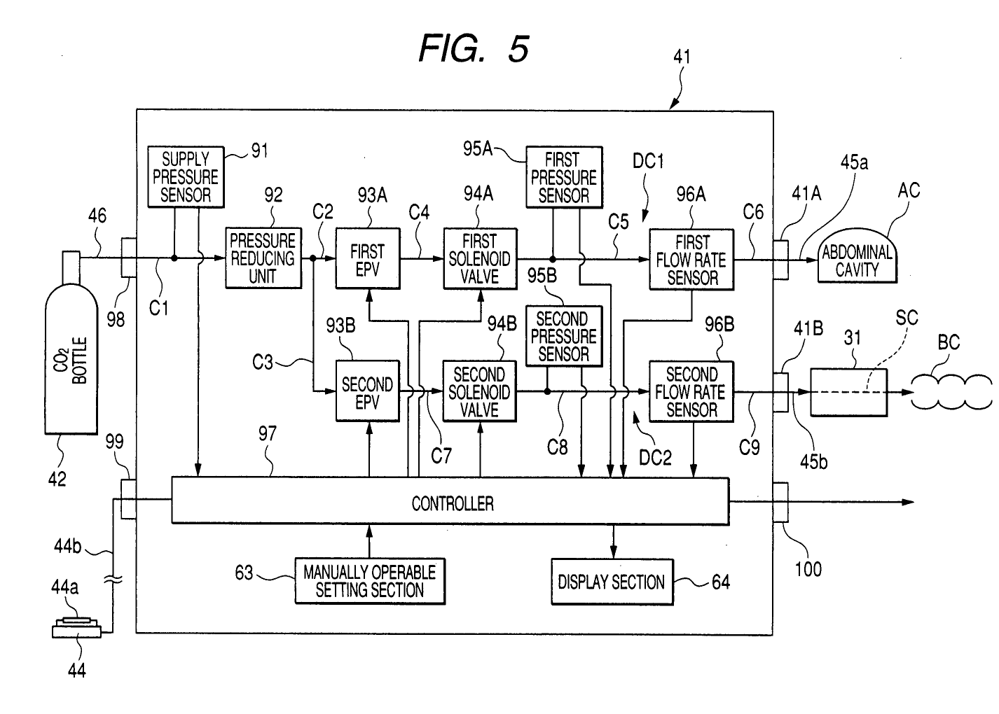

Reference numerals 14, 15, and 16 designate first, second, and third trocars, which are inserted into, for example, an abdominal portion of the patient 10, respectively. The first trocar 14 allows an endoscope, described herein after, of the first endoscope system 2 to be guided into a first body cavity, such as an abdominal cavity AC (see FIG. 5) of the patient 10. The abdominal cavity AC, which means a cavity separated by the diaphragm from the thoracic cavity above and by the plane of the pelvic inlet from the pelvic cavity below, serves as a first body cavity of the patient 10 according to the first embodiment.

The second trocar 15 permits guide of a treatment tool into the abdominal cavity AC. The treatment tool, such as the electric scalpel 13, is operative to remove and/or treat a tissue corresponding to at least one site to be treated in the abdominal cavity AC.

The third trocar 16 allows predetermined gas for the abdominal cavity, such as carbon dioxide gas, to be introduced into the abdominal cavity AC. The carbon dioxide gas, referred to as “CO₂” can be easily absorbed into a living body, such as the patient 10, which is supplied from the gas supply system 4. The carbon dioxide gas can be introduced into the inside of the abdominal cavity AC through at least one of the trocars 14 and 15.

The first endoscope system 2 includes a rigid endoscope 21 as a first endoscope with, for example, a rigid insert portion at one end thereof.

The rigid endoscope 21 is referred to as “rigidscope” hereinafter. The first endoscope system 2 includes a first light source 22, a first camera control unit 23, referred to as “first CCU” hereinafter, and a camera (TV camera) 24 for endoscopes. The first endoscope system 2 includes a camera for endoscopes.

One end portion of the insertion portion (not shown) of the rigidscope 21, for example, is configured to be inserted in part into the first trocar 14. The rigidscope 21 is provided with an illumination optics (not shown) and an observation optics (not shown), which are installed in the one end portion of the insertion portion. The illumination optics is composed of, for example, a light guide and the like, and configured to illuminate light onto a target, such as the site to be treated, of the inside of the patient 10. For example, the observation optics is composed of relay lenses and the like. The observation optics is configured to optically deliver an optical image of the target illuminated by the light.

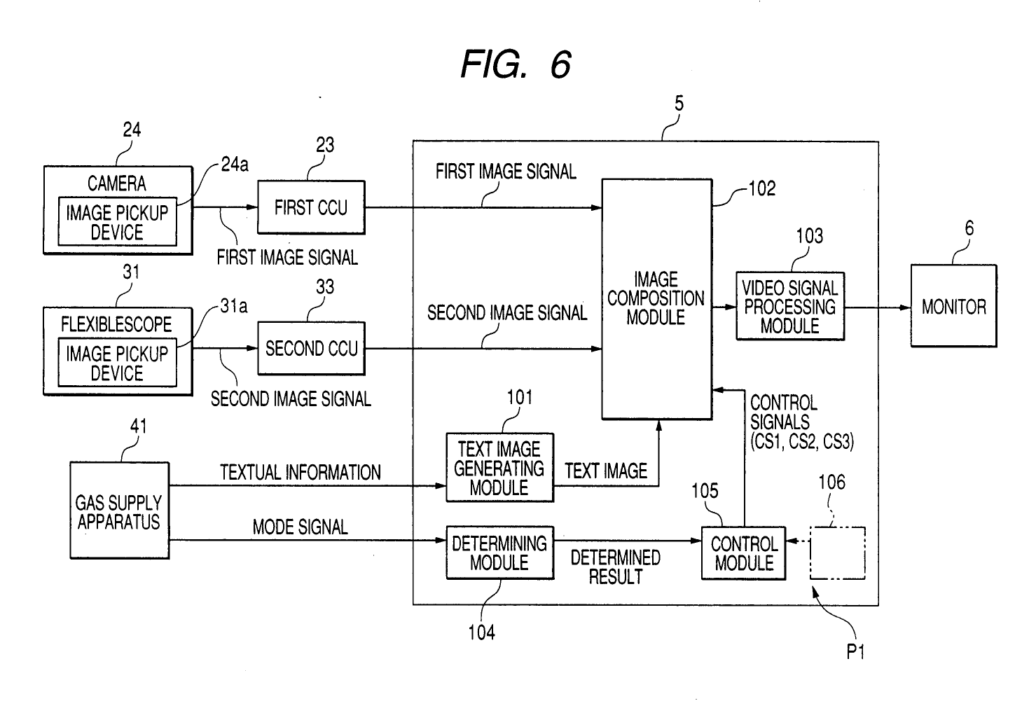

The rigidscope 21 is provided at the other end side of the insertion portion with an eyepiece 25 that allows an operator to observe the optical image delivered by the observation optics. The camera 24 is detachably installed in the eyepiece 25. The camera 24 is integrated with an image pickup device 24a, such as a CCD (Charge Coupled Device) or the like (see FIG. 6), having a light sensitive pixel area, wherein the optical image delivered by the observation optics is focused on the light sensitive pixel area thereof. The optical image of the target focused on the light sensitive pixel area of the image pickup device 24a is photoelectrically converted into an electric signal as a first image signal, by the image pickup device.

The first endoscope system 2 is provided with a light guide cable 26 extending from one side of the other end of the rigidscope 21. The light guide cable 26 is optically coupled to the first light source 22, allowing optical coupling between the rigidscope 21 and the first light source 22. The first endoscope system 2 is provided with an image pickup cable 27 electrically connecting between the first CCU 23 and the camera 24.

The first light source 22 has a function of supplying illumination light to the illumination optics of the rigidscope 21 via the light guide cable 26. The first CCU 23 is operative to execute electrical drive control of the image pickup device 24a of the camera 24. When the first image signal corresponding to the optical image of the target, which is picked up by the image pickup device 24a, is sent to the first CCU 23, the first CCU 23 is operative to receive the first image signal to subject the received first image signal to image processing of necessity. The first CCU 23 is operative to output the image-processed first image signal to the system controller 5. Image processing of the system controller 5 with respect to the first image signal allows at least one of the monitor 6 and the center display panel 7 to display a second image of the target thereon based on the image-processed first image signal.

The first image is a first endoscopic image, referred to also as “abdominal-cavity image” corresponding to the first image signal picked up by the rigidscope 21.

The second endoscope system 3 includes a flexible endoscope 31 as a second endoscope with, for example, a flexible insert portion 34 at one end thereof. The flexible insert portion is so flexible that it can be inserted into a lumen BC as a second body cavity of the patient. In the specification, the lumen is defined as the cavity of an organ in a patient, such as the cavity of the stomach, the cavity of the large intestine, the cavity of a blood vessel, or the like in the patient. The flexible endoscope 31 is referred to as “flexiblescope” hereinafter. The second endoscope system 3 includes a second light source 32, and a second CCU 33.

The flexiblescope 31 has a substantially hollow-rod (tubular) shape, which is narrow in diameter and flexible. The flexiblescope 31 is internally formed with a gas delivery channel SC (see FIG. 5).

Specifically, the flexiblescope 31 is provided at its one end with the insert portion 34 to be inserted at its one end into the interior of the lumen BC, and a manipulator 35 whose one end is joined to the other end of the insert portion 34. The manipulator 35 allows, for example, an operator to manipulate the flexiblescope 31. The flexiblescope 31 is provided with a universal cord 36 whose one end is joined to the other end of the manipulator 35.

The manipulator 35 is provided with a gas and water supply switch 35a mounted thereon. The gas and water supply switch 35a is formed with a through hole, also referred to as “gas and water supply channel), communicated with the gas delivery channel SC inside of the manipulator 35. The gas and water supply switch 35a, the gas delivery channel SC, and the insert portion 34 allow the operator to supply gas and water therethrough.

It should be noted that the term “operator” through the specification is not necessarily limited to a person who actually treats; the term “operator” refers to a concept that involves any of nurses or other operators who assist such a treatment action.

The manipulator 35 is provided with a suction switch 35b disposed thereto and a flexion knob 37 that allows the operator to flex a flexible portion (not shown) of the flexiblescope 31. The manipulator 35 is formed with a treatment tool channel communicated with the gas delivery channel SC, and the flexiblescope 31 is provided with a treatment tool insertion opening 38 formed to be communicated with the treatment tool channel in the manipulator 35. The treatment tool insertion opening 38 allows treatment tools to be inserted therethrough. The other end of the universal cord 36 is coupled to a light source connector 36a optically detachably.

The second light source 32 has a connector 30 such that the universal cord 36 is optically coupled to the second light source 32 through the light source connector 36a and the connector 30.

Specifically, the second light source 32 has a function of supplying illumination light to the flexiblescope 31 through the connector 30, the light source connector 36a, and the universal cord 36.

The flexiblescope 31 is provided at its one end of the insertion portion 34 with an illumination optics. The illumination optics is composed of a light guide that can illuminate light on a target inside the patient 10, such as the lumen BC, through an illumination window disposed to one side of the one end of the insertion portion 34.

The flexiblescope 31 is provided with an image pickup device 31a (see FIG. 6), such as a CCD (Charge Coupled Device) or the like, installed in the one end of the insertion portion 34. The image pickup device 31a has a light sensitive pixel area. The image pickup device 31a is so arranged that an optical image of the target illuminated by the light outputted from the illumination optics can be focused on the light sensitive pixel area of the image pickup device 31a.

The image pickup device 31a of the flexiblescope 31 is electrically connected to the second CCU 33 through the universal cord 36 and the like. Reference numeral 39 is an electric cable electrically connecting between an electric connector 36b attached to the light source connector 36a and the second CCU 33.

The image pickup device 31a is operative to photoelectrically convert the optical image of the target focused on the light sensitive pixel area into an electric signal as a second image signal.

The second CCU 33 is operative to execute electrical drive control of the image pickup device 31a. When the second image signal corresponding to the optical image of the target, which is picked up by the image pickup device 31a, is sent to the second CCU 33 through the electric cable 39, the second CCU 33 is operative to receive the second image signal to subject the received first image signal to image processing of necessity. The second CCU 33 is operative to output the image-processed second image signal to the system controller 5. Image processing of the system controller 5 with respect to the second image signal allows at least one of the monitor 6 and the center display panel 7 to display a second image of the target thereon based on the image-processed second image signal. That is, the second image is an endoscopic image, referred to also as “lumen image”, corresponding to the second image signal picked up by the flexiblescope 31.

Turning now to the gas supply system 4, it includes a gas supply apparatus 41, a carbon dioxide gas cylinder (CO₂ bottle) 42 as a supplier, a foot switch 44 serving as an operation switch for controlling supply of the carbon dioxide gas into the lumen BC, an abdominal cavity tube 45a, and a lumen tube 45b. The CO₂ bottle 42 has stored carbon dioxide in liquid.

The gas supply apparatus 41 is provided with a first adapter (connector) 41A for insufflation of the abdominal cavity AC and a second adapter 41B for insufflation of the lumen BC. The first adapter 41A is airtightly coupled to one end of the abdominal cavity tube 45a. The other end of the abdominal cavity tube 45a is airtightly coupled to the third trocar 16. The second adapter 41B is airtightly coupled to one end of the lumen tube 45b. The other end of the lumen tube 45b is airtightly coupled to a tube coupler 43a formed on one side of the adapter 43, which allows the lumen tube 45b to be communicated with the gas delivery channel SC inside the flexiblescope 31 through the adapter 43.

The foot switch 44 is provided with a switch portion 44a and is configured to provide instructions to instruct supply of the carbon dioxide gas into the lumen BC to the gas supply apparatus 41 while the operator or the like depresses the switch portion 44a with operator's foot or the like.

The gas supply apparatus 41 and the CO₂ bottle 42 are coupled to each other through a high-pressure gas tube 46. The gas supply apparatus 41 and the foot switch 44 are electrically connected to each other through a foot switch cable 44b. The electrical connection between the foot switch 44 and the gas supply apparatus 41 can be established by wireless. Each of the tubes 45a and 45b is made of a material such as, for instance, silicone, Teflon®, or other similar materials.

The system controller 5 is operative to perform control of the whole system 1. With the system controller 5, the center display panel 7, the center operation panel 8, and peripheral devices including the electric scalpel device 12, the first light source 22, the second light source 32, the first CCU 23, the second CCU 33, and the gas supply apparatus 41 are communicably connected through communication buses (not shown), respectively.

The monitor 6 has a function of receiving the first image signal and/or second image signal outputted from the first and second CCUs 23 and 33 to display at least one of the first image and/or second image thereon based on the received first image signal and/or second image signal.

The center display panel 7 is composed of a display screen, such as a liquid crystal screen or the like and is electrically connected to the system controller 5. The center display panel 7 allows concentrative display of operating states of the peripheral devices together with the first and second images on the display screen.

The center operation panel 8 is composed of a display section, such as a liquid crystal screen or the like, and a touch-sensitive device integrally formed on the display section. The display section of the center operation panel 8 has a display function of providing a setting screen on which operable switches (buttons) for the peripheral devices are graphically displayed. The display section has an operating function that allows the operator to operate the operable switches by touching them.

The center operation panel 8 is electrically connected to the system controller 5.

Specifically, the operator touches at least one of the operable switches with, for example, a finger, so that the touch-sensitive device sets operating conditions corresponding to at least one of the touched operable switches to remotely send to the system controller 5 instructions for operating a corresponding one of the peripheral devices based on the set operating conditions. These remote operations of the graphical operable switches on the center operation panel 8 with respect to the peripheral devices are substantially identical to direct operations of operable switches directly attached to the peripheral devices.

The peripheral devices including the electric scalpel device 12, the first and second light sources 22 and 32, the first and second CCUs 23 and 33, the gas supply apparatus 41, and a VTR (Video Tape Recorder), which is not shown, are mounted on the cart 9. In addition, the system controller 5, the center display panel 7, and the center operation panel 8 are mounted on the cart 9.

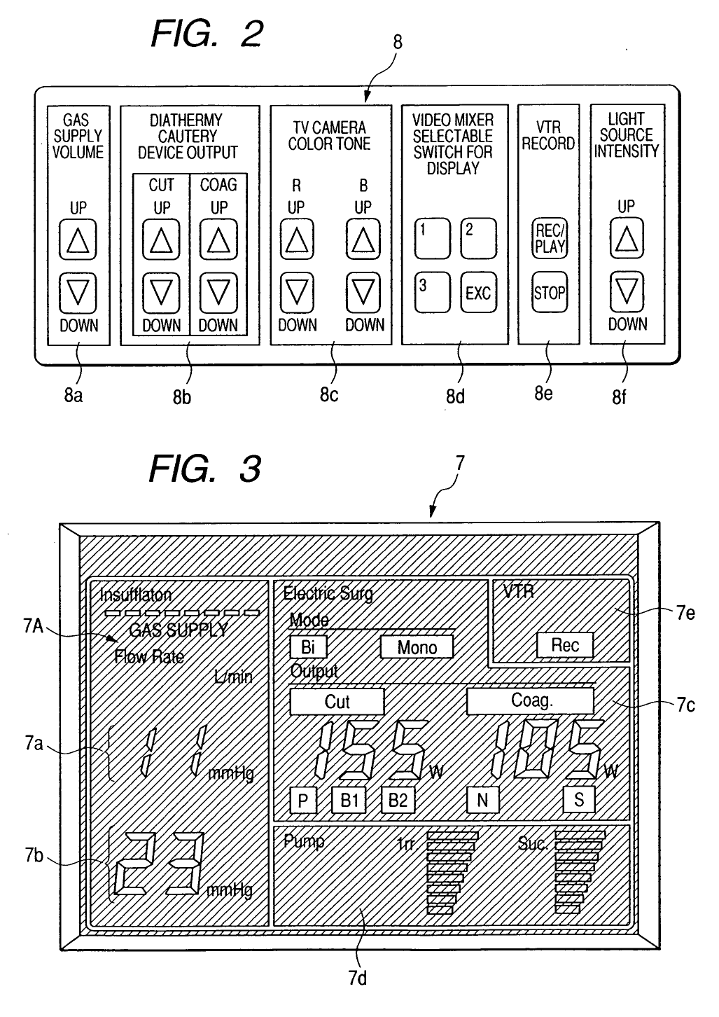

A configuration example of the operation panel 8 is illustrated in FIG. 2.

The operation panel 8 is composed of a display screen, such as a liquid crystal display, and a touch-sensitive device integrally formed on the display screen. On the display screen, manually operable sections, such as manually operable graphical buttons, are displayed. The manually operable sections allow the operator to set operating conditions (parameters) with respect to the peripheral devices to give instructions for operating them based on the set operating conditions to the system controller 5 or the corresponding peripheral devices.

Specifically, the operator touches at least one of the operable sections (operable buttons), with, for example, a finger so that the touch-sensitive device sets operating conditions corresponding to at least one of the touched operable sections to send to the system controller 5 instructions for operating the corresponding one of the peripheral devices based on the set operating conditions. The system controller 5 controls the corresponding one of the peripheral devices based on the instructions so that the corresponding one of the peripheral devices operates under the set operating conditions.

For example, as shown in FIG. 2, manual operation buttons 8a are graphically displayed on the display screen of the operation panel 8. The manual operation buttons 8a allow the operator to adjust the flow-rate of carbon dioxide gas supplied to the abdominal cavity AC or the lumen BC from the gas supply apparatus 41.

Manual operation buttons 8b are graphically displayed on the display screen of the operation panel 8. The manual operation buttons 8b permit the operator to adjust an output value of the electric scalpel device 12. Manual operation buttons 8c are graphically displayed on the display screen of the operation panel 8. The manual operation buttons 8c allow the operator to control color tones of the first and second CCUs 23 and 33.

In addition, manual operation buttons 8d are graphically displayed on the display screen of the operation panel 8. The manual operation buttons 8d allow the operator to send instructions to the system controller 5 for selectively switching the first image (the endoscopic image of the rigidscope 21) and the second image (the endoscope image of the flexiblescope 31), which are displayed on the monitor 6.

Manual operation buttons 8e are graphically displayed on the display screen of the operation panel 8. The manual operation buttons 8e allow the operator to send instructions to the system controller 5 for making the VTR start recording the first image and/or second image on a video tape or for stopping the record of the first image and/or second image thereon.

Manual operation buttons 8f are graphically displayed on the display screen of the operation panel 8. The manual operation buttons 8f permit the operator to adjust light intensity of the illumination light irradiated from the first light source 22 and that of the illumination light irradiated from the second light source 32.

An example of the display panel 7 shown in FIG. 1 is illustrated in FIG. 3.

As illustrated in FIG. 3, display areas 7A (7a, 7b), 7c, 7d, and 7e are graphically represented on the display screen of the display panel 7. The display areas 7A (7a, 7b), 7c, 7d, and 7e are allocated to the gas supply apparatus 41, the electric scalpel device 12, a water pump (not shown), and the VTR, which are communicated to be controlled by the system controller 5, respectively.

The current settings of the peripheral devices and the operating states thereof are displayed on the corresponding display areas 7A, (7a, 7b), 7c, 7d and 7e, respectively. For example, the display area 7A is operative to display the settings and the operating state of the gas supply apparatus 41. Specifically, the display area 7A includes a display area 7a on which a current pressure inside the lumen BC of the patient 10 is displayed, and a display area 7b on which a current pressure inside the abdominal cavity AC of the patient 10 is displayed. The display area 7A also includes display areas for displaying the flow-rate (Flow Late) of the carbon dioxide gas supplied from the gas supply apparatus 41 and the volume (GAS SUPPLY) of the carbon dioxide gas remaining in the CO₂ bottle 42.

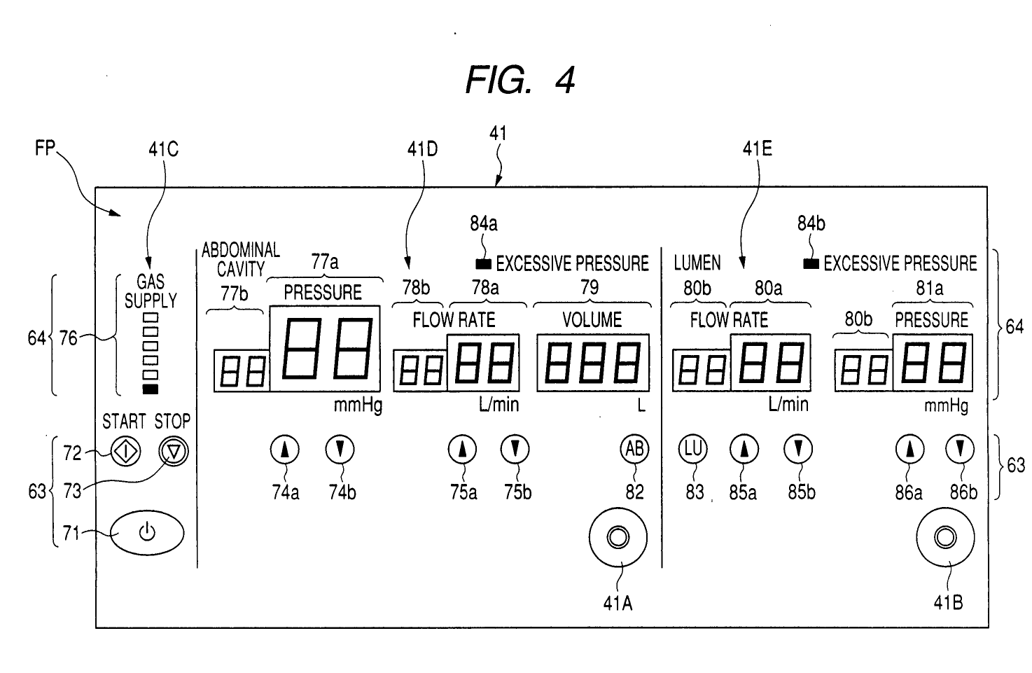

Next, a configuration example of the manually operable setting section 63 and the display section 64 provided on a front panel FP of the gas supply apparatus 41 is described with reference to FIG. 4. In the first embodiment, for example, the front panel FP is attached along one side of a housing of the gas supply apparatus 41.

As shown in FIG. 4, the manually operable setting section 63 and the display section 64 are graphically displayed on the front panel FP of the gas supply apparatus 41. The manually operable setting section 63 and display section 64 are divided in, for instance, three graphical setting and display sections 41C to 41E.

The setting and display section 41C serves as a supply source setting and display section that allows the operator to enter instructions related to the carbon dioxide gas supplied from the CO₂ bottle 42. In addition, the setting and display section 41C is designed to display the state of carbon dioxide gas supplied from the CO₂ bottle 42.

The setting and display section 41D serves as a setting and display section for an abdominal cavity. Specifically, the setting and display section 41D allows the operator to set parameters related to the pressure inside the abdominal cavity AC and the carbon dioxide gas insufflation thereof. The setting and display section 41D allows the operator to enter instructions related to the pressure inside the abdominal cavity AC and the carbon dioxide gas insufflation thereof. The setting and display section 41D is designed to display the state of the abdominal cavity AC depending on the carbon dioxide gas being insufflated thereinto.

The setting and display section 41E serves as a setting and display section for a lumen. Specifically, the setting and display section 41E allows the operator to set parameters related to the carbon dioxide gas insufflation of the lumen BC; the setting and display section 41E is designed to display the state of the lumen BC depending on the carbon dioxide gas being insufflated thereinto.

The first adaptor 41A is attached to the lower side of the setting and display section 41D of the front panel FP; the second adaptor 41B is attached to the lower side of the setting and display section 41E of the front panel FP.

The setting and display section 41C is provided with a power switch 71, a gas-supply start button 72, and a gas-supply stop button 73a as the manually operable setting section 63. In addition, the setting and display section 41C is provided with a gas remaining volume indicators 76 as the display section 64.

The setting and display section 41D is provided with pressure displays 77a and 77b for the pressure inside the abdominal cavity AC, flow-rate displays 78a and 78b for the abdominal cavity AC, a total volume display 79 for the abdominal cavity AC, and an excessive pressure indicator 84a for the abdominal cavity AC as the display section 64.

The setting and display section 41D is provided with pressure setting buttons 74a and 74b for the pressure inside the abdominal cavity AC, flow-rate setting buttons 75a and 75b for the abdominal cavity AC, and an abdominal cavity select button 82 (see “AB” in FIG. 4) as the manually operable setting section 63.

The setting and display section 41E is provided with flow-rate displays 80a and 80b for the lumen BC as the display section 64. The setting and display section 41E is provided with pressure displays 81a and 81b for the lumen BC, and an excessive pressure indicator 84b for the lumen BC as the display section 64.

The setting and display section 41E is provided with a lumen select button 83 (see “LU” in FIG. 4), flow-rate setting buttons 85a and 85b for the lumen BC, and pressure setting buttons 86a and 86b as the manually operable setting section 63.

The power switch 71 serves as a switch that permits the operator to turn power on and off to the apparatus 41. The gas-supply start button 72 serves as a button that allows the operator to send an instruction to start the supply of the carbon dioxide gas into the abdominal cavity AC to a controller 97 described hereinafter. The gas-supply stop button 73 serves as a button that permits the operator to send an instruction to stop the supply of the carbon dioxide gas to the controller 97.

The pressure setting buttons 74a and 74b serve as buttons that allow the operator to send instructions to change the corresponding parameter (the pressure inside the abdominal cavity AC) to a pressure setting. The flow-rate setting buttons 75a and 75b serve as buttons that enable the operator to send instructions to change the corresponding parameter (the flow-rate of the carbon dioxide gas being insufflated into the abdominal cavity AC) to a flow-rate setting. The flow-rate setting buttons 85a and 85b serve as buttons that permit the operator to send instructions to change the corresponding parameter (the flow-rate of the carbon dioxide gas being insufflated into the lumen BC) to a flow-rate setting. The pressure setting buttons 86a and 86b serve as buttons that permit the operator to send instructions to change the corresponding parameter (the pressure inside the lumen BC) to a pressure setting.

Specifically, the pressure setting buttons include an up button 74a and a down button 74b. Every time the operator clicks the up button 74a, the pressure setting inside the abdominal cavity AC turns up; every time the operator clicks the down button 74b, the pressure setting turns down. The pressure setting variably determined by the up and down buttons 74a and 74b is sent to the controller 97 every time at least one of the up and down buttons 74a and 74b is operated.

Similarly, the flow-rate setting buttons include an up button 75a and a down button 75b. The flow-rate setting of the carbon dioxide gas to be insufflated into the abdominal cavity AC turns up every time the operator clicks the up button 75a; the flow-rate setting turns down every time the operator clicks the down button 75b. The flow-rate setting variably set by the up and down buttons 75a and 75b is sent to the controller 97 every time at least one of the up and down buttons 75a and 75b is operated.

Furthermore, the flow-rate setting buttons include an up button 85a and a down button 85b. Every time the operator clicks the up button 85a, the flow-rate setting turns up; every time the operator clicks the down button 85b, the flow-rate setting turns down. The flow-rate setting variably determined by the up and down buttons 85a and 85b is sent to the controller 97 every time at least one of the up and down buttons 85a and 85b is operated.

The pressure setting buttons include an up button 86a and a down button 86b. The pressure setting inside the lumen BC turns up every time the operator clicks the up button 86a; the pressure setting turns down every time the operator clicks the down button 86b. The pressure setting variably set by the up and down buttons 86a and 86b is sent to the controller 97 every time at least one of the up and down buttons 86a and 86b is operated.

The gas remaining volume indicators 76 are vertically arranged so that a top indicator that is lighting indicates the amount of carbon dioxide gas available.

The right-side pressure display 77a is configured to display a pressure value (in mmHg) based on a measured value of a first pressure sensor 95A described hereinafter. The left-side pressure display 77b is configured to display the pressure setting determined based on the operations of, for example, the pressure setting buttons 74a and 74b. The right-side flow-rate display 78a is configured to display a flow-rate (in L/min) based on a measured value of a first flow-rate sensor 96A described hereinafter. The left-side flow-rate display 78b is configured to display the flow-rate setting determined based on the operations of, for example, the flow-rate setting buttons 75a and 75b.

The total volume display 79 is configured to display a total amount of carbon dioxide gas calculated by the controller 97 based on the measured value of the first flow-rate sensor 96A.

The right-side flow-rate display 80a is configured to display a flow-rate (in L/min) based on a measured value of a second flow-rate sensor 96B described hereinafter. The left-side flow-rate display 80b is configured to display the flow-rate setting determined based on the operations of, for example, the flow-rate setting buttons 85a and 85b.

The right-side pressure display 81a is configured to display a pressure (in mmHg) based on a measured value of a second pressure sensor 95B described hereinafter. The left-side pressure display 81b is configured to display the pressure setting determined based on the operations of, for example, the pressure setting buttons 86a and 86b.

When the operator turns on the abdominal cavity select button 82, the button 82 is configured to send to the controller 97 an instruction to make it execute operations for supplying the carbon dioxide gas into the abdominal cavity AC. In other words, when the operator turns on the abdominal cavity select button 82, the button 82 is configured to send to the controller 97 an instruction to change the operation mode thereof to an abdominal cavity insufflation mode.

When the operator turns on the lumen select button 83, the button 83 is configured to send to the controller 97 an instruction to make it execute operations for supplying the carbon dioxide gas into the lumen BC. In other words, when the operator turns on the lumen select button 83, the button 83 is configured to send to the controller 97 an instruction to change the operation mode thereof to a lumen insufflation mode.

The excessive pressure indicator 84a consists of, for example, red LED (light emitting diode). The excessive pressure indicator 84a is configured to turn on or flash on and off based on a control signal sent from the controller 97 at anytime the pressure measured by the first pressure sensor 96A exceeds a threshold value of the pressure inside the abdominal cavity AC by a predetermined pressure. The turning-on or the flashing of the excessive pressure indicator 84a allows the operator to visually recognize that the pressure inside the abdominal cavity AC exceeds the threshold value by the predetermined pressure or more.

The excessive pressure indicator 84b consists of, for example, red LED. The excessive pressure indicator 84b is configured to turn on or flash on and off based on a control signal sent from the controller 97 at anytime the pressure measured by the second pressure sensor 96B exceeds a threshold value of the pressure inside the lumen BC by a predetermined pressure. The turning-on or the flashing of the excessive pressure indicator 84b allows the operator to visually recognize that the pressure inside the lumen BC exceeds the threshold value by the predetermined pressure or more.

The center operation panel 8 allows the operator to set the parameters of the gas supply apparatus 41, which include the setting of the pressure inside the abdominal cavity AC, and the settings of the flow-rates for the abdominal cavity AC and the lumen BC. Specifically, the settings determined on the center operation panel 8 for the corresponding parameters are sent to the controller 97 through the system controller 5. The controller 97 carries out abdominal-cavity pressure control, lumen pressure control, abdominal-cavity flow-rate control, and lumen flow-rate control based on the corresponding parameters, respectively.

In addition, the center display panel 7 can be configured to display at least one of the settings, which has been specified by the operator, displayed on the pressure displays 77a, 77b, 81a and 81b, flow-rate displays 78a, 78b, 80a, and 80b, and the total volume display 79.

Specifically, the controller 97 operates to send at least one of the settings, which has been specified by the operator, displayed on he pressure displays 77a, 77b, 81a and 81b, flow-rate displays 78a, 78b, 80a, and 80b, and the total volume display 79 to the system controller 5. The system controller 5 receives at least one of the settings sent from the controller 97 to display it on the center display panel 7.

The structures of the manually operable setting section 63 and the display section 64 in the front panel FP allow the operator to easily give instructions to the controller 97 and to easily visually recognize the parameters related to the abdominal cavity AC and the lumen BC.

Next, a structure of the gas supply apparatus 41 will be described hereinafter with reference to FIG. 5.

As shown in FIG. 5, the gas supply apparatus 41 includes first to ninth delivery channels C1 to C9, a supply pressure sensor 91, and a pressure reducing unit 92. The gas supply apparatus 41 includes first and second electropneumatic proportional valves (EPVs) 93A and 93B as examples of pressure regulating valves, serving as a pressure regulator.

In addition, the gas supply apparatus 41 includes first and second electromagnetic valves (solenoid valves) 94A and 94B as examples of open/close valves. The first and second electromagnetic valves 94A and 94B, for example, serve as the pressure regulator.

The gas supply apparatus 41 includes the first and second pressure sensors 95A and 95B, the first and second flow-rate sensors 96A and 96B, and the controller 97. In addition, the gas supply apparatus 41 includes a high pressure adapter 98, a connector 99 for switches, a communication connector 100, the manually operable setting section 63, the display section 64, and the first and second adapters 41A and 41B.

Specifically, the CO₂ bottle 42 has a discharge port (cock) to which one end of the high-pressure gas tube 46 is joined. The other end of the high-pressure gas tube 46 is joined to the high-pressure adapter 98. The high-pressure adapter 98 is joined to an inlet of the pressure reducing unit 92 via the first delivery channel C1. The supply pressure sensor 91 is attached to the first delivery channel C1. An outlet of the pressure reducing unit 92 is branched into the second delivery channel C2 for the abdominal cavity AC and the third delivery channel C3 for the lumen BC.

One branched channel C2 is coupled to an inlet of the first electropneumatic proportional valve 93A. An outlet of the first electropneumatic proportional valve 93A is coupled to an inlet of the first solenoid valve 94A through the fourth delivery channel C4. An outlet of the first solenoid valve 94A is coupled to the fifth delivery channel C5 to which the first pressure sensor 95A is attached. The fifth delivery channel C5 is coupled to an inlet of the first flow rate sensor 96A whose outlet is coupled through the sixth delivery channel C6 and the first adapter 41A to the one end of the abdominal cavity tube 45a.

The other end of the tube 45a is coupled to the third trocar 16, and the third trocar 16 is inserted into the abdominal cavity AC of the patient 10.

The other branched channel C3 is coupled to an inlet of the second electropneumatic proportional valve 93B. An outlet of the second electropneumatic proportional valve 93B is coupled to an inlet of the second solenoid valve 94B through the seventh delivery channel C7. An outlet of the second solenoid valve 94B is coupled to the eighth delivery channel C8 to which the second pressure sensor 95B is attached. The eighth delivery channel C8 is coupled to an inlet of the second flow rate sensor 96B whose outlet is coupled through the ninth delivery channel C9 and the second adapter 41B to the one end of the lumen cavity tube 45b.

The other end of the tube 45b is communicably coupled to the gas delivery channel SC formed inside the flexiblescope 31 through the tube coupler 43a, and the insertion portion 34 of the flexiblescope 31 is inserted into the lumen BC of the patient 10.

In the first embodiment, the first electropneumatic proportional valve 93A, the fourth delivery channel C4, the first solenoid valve 94A, the fifth delivery channel C5, the first flow-rate sensor 95A, the sixth delivery channel C6, the first adapter 41A, and the abdominal cavity tube 45a constitute a first CO₂ supply path DC1. The first CO₂ supply path DC1 directs the carbon dioxide gas into the abdominal cavity AC.

Similarly, the second electropneumatic proportional valve 93B, the channel C7, the second solenoid valve 94B, the channel C8, the second flow-rate sensor 96B, the channel C9, the second adapter 41B, the tube coupler 43a, the lumen tube 45b, and the gas delivery channel SC formed inside the flexiblescope 31 constitute a second CO₂ supply path DC2. The second CO₂ supply path DC2 is configured to direct the carbon dioxide gas into the lumen BC.

The gas supply apparatus 41 has the foot switch cable 44b electrically connected to the switch connector 99; the foot switch cable 44b is electrically connected to the foot switch 44. The switch connector 99 is electrically connected to the controller 97. With the electrical connection between the foot switch 44 and the controller 97, the depressing operation of the switch portion 44a by the operator allows the instruction to be provided through the foot switch cable 44b to the controller 97. Incidentally, communications between the foot switch 44 and the controller 97 can be wirelessly established. The manually operable section 63 and the display section 64 are electrically connected to the controller 97.

The supply pressure sensor 91 is electrically connected to the controller 97. The supply pressure sensor 91 has a function of detecting the pressure of the carbon dioxide gas flowing from the CO₂ bottle 42 to the first delivery channel C1 to send the detected result (detected pressure value) to the controller 97. The pressure reducing unit 92 is configured to reduce in pressure the carbon dioxide gas such that the carbon dioxide gas has a predetermined pressure. Thereafter, the carbon dioxide gas is guided via the second delivery channel C2 to the first electropneumatic proportional valve 93A.

Each of the first and second electropneumatic proportional valves 93A and 93B is provided with a solenoid composed of, for example, a magnet coil (solenoid coil) and a compass needle, which are not shown. Each of the first and second electropneumatic proportional valves 93A and 93B is provided with a thin film for pressure control, and a pressure reducing spring. The solenoid is electrically connected to the controller 97. Each of the first and second electropneumatic proportional valves 93A and 93B is configured such that the solenoid controls force applied on the thin film by the pressure reducing spring depending on a control signal applied from the controller 97, thereby regulating the pressure of the carbon dioxide gas.

Specifically, the first electropneumatic proportional valve 93A is designed to change its opening in proportional to a voltage or a current as the control signal applied from the controller 97 so as to regulate the pressure and the flow-rate of the carbon dioxide gas flowing therethrough within corresponding appropriate ranges, respectively

For example, the appropriate range of the pressure of the carbon dioxide gas to be insufflated into the abdominal cavity AC is preferably 0 to 80 mmHg or thereabout; the appropriate range of the flow-rate thereof to be insufflated thereinto is preferably 0.1 to 35 L/min or thereabout.

The second electropneumatic proportional valve 93B is designed to change its opening in proportional to a voltage or a current as the control signal applied from the controller 97 so as to regulate the pressure and the flow-rate of the carbon dioxide gas flowing therethrough within corresponding appropriate ranges, respectively.

For example, the appropriate range of the pressure of the carbon dioxide gas to be insufflated into the lumen BC is preferably 0 to 500 mmHg or thereabout; the appropriate range of the flow-rate thereof to be insufflated thereinto is preferably 1 to 3 L/min or thereabout.

Each of the first and second solenoid valves 94A and 94B is electrically connected to the controller 97 and configured to open and close based on control signals sent from the controller 97. The opening and closing of the first solenoid valve 94A allow first CO₂ supply path DC1 to open and close, respectively. Similarly, the opening and closing of the second solenoid valve 94B permit the second CO₂ supply path DC2 to open and close, respectively.

The first pressure sensor 95A has a function of measuring a pressure in the fifth delivery channel C5, in other words, a pressure inside the abdominal cavity AC, thereby sending the measured result to the controller 97.

The second pressure sensor 95B is electrically connected to the controller 97. The second pressure sensor 95B has a function of measuring a pressure in the eighth delivery channel C8, in other words, a pressure inside the lumen BC thereby sending the measured result to the controller 97.

The first and second flow rate sensors 96A and 96B are electrically connected to the controller 97. The first flow rate sensor 96A has a function of detecting the flow rate of the carbon dioxide gas flowing through the first solenoid valve 94A and the fifth delivery channel C5. Similarly, the second flow rate sensor 94B is operative to detect the flow rate of the carbon dioxide gas flowing through the second solenoid valve 94B and the eighth delivery channel C8. Each of the first and second flow rate sensors 96A and 96B is configured to send the detected result to the controller 97.

The controller 97 is operative to receive the measured values outputted from the supply pressure sensor 91, the first and second pressure sensors 95A and 95B, the first and second flow rate sensors 96A and 96B. The controller 97 is configured to execute opening control (pressure control) of the first electropneumatic proportional valve 93, opening and closing controls of each of the first and second solenoid valves 94A and 94B, and display control of the display section 64 based on the received measured values.

Specifically, the controller 97 is configured to execute opening control of the first electropneumatic proportional valve 93A in an abdominal-cavity insufflation mode during which the abdominal cavity select button 82 is in the ON position. The opening control of the first electropneumatic proportional valve 93A permits the pressure of the carbon dioxide gas being insufflated into the abdominal cavity AC to be regulated within the appropriate range of 0 to 80 mmHg or thereabout, and the flow-rate thereof to be regulated within 0.1 to 35 L/min or thereabout.

Similarly, the controller 97 is configured to execute opening control of the second electropneumatic proportional valve 93B in a lumen insufflation mode during which the lumen select button 83 is in the ON position. The opening control of the second electropneumatic proportional valve 93B permits the pressure of the carbon dioxide gas being insufflated into the lumen BC to be regulated within the appropriate range of 0 to 500 mmHg or thereabout, and the flow-rate thereof to be regulated within 0.1 to 35 L/min or thereabout.

In the structure of the gas supply apparatus 41, when the cock of the CO₂ bottle 42 is opened, carbon dioxide stored therein in a liquid form is vaporized to form the carbon dioxide gas. The carbon dioxide gas is delivered to the pressure reducing unit 92 through the high-pressure gas tube 46, the high pressure adapter 98, and the first delivery channel C1 of the gas supply apparatus 41. The carbon dioxide gas is reduced in pressure by the pressure reducing unit 92 to have a predetermined pressure

Thereafter, the carbon dioxide gas is supplied into either the abdominal cavity AC through the first CO₂ supply path DC1 or the lumen BC through the second CO₂ supply path DC2 based on the control signals sent from the controller 97.

Incidentally, in the first embodiment, the channels and the like constituting the first CO₂ supply path DC1 provide airtight junction therebetween, and the channels and the like constituting the second CO₂ supply path DC2 provide airtight junction therebetween.

In the first embodiment, as shown in FIG. 1, the adapter 43 corresponds to the communicable connecting location of the lumen tube 45b with respect to the gas delivery channel SC inside the manipulator 35. This configuration allows the adapter 43 to be arranged at a position closer to the insertion section 34 than the gas and water supply switch 35a through which the through hole is formed.

Specifically, in the first embodiment, the through hole of the gas and water supply switch 35a of the manipulator 35 of the flexiblescope 31 deviates from the second CO₂ supply path DC2 including the lumen tube 45b through which the carbon dioxide gas is supplied. Thus, in the first embodiment, the operator is able to perform the operations to supply the carbon dioxide gas into the lumen BC and to interrupt the supply thereof by the operations to depress the switch portion 44a of the foot switch 44 and release it without opening and closing the through hole in the switch 35a.

In the gas supply apparatus 41 according to the first embodiment, a relief valve (opening and closing valve) can be provided at the midstream of the sixth delivery channel C6 between the first flow rate sensor 96A and the first adapter 41A. In this modification, the relief valve is electrically connected to the controller 97. The relief valve is operative to remain in a closed state, and to open based on a control signal sent from the controller 97 when the measured value of the first pressure sensor 95A exceeds the predetermined threshold value. Like the abdominal cavity side, a relief valve can be provided at the midstream of the ninth delivery channel C9 between the second flow rate sensor 96B and the second adapter 41B. In this case, the relief valve is electrically connected to the controller 97. The relief valve is operative to remain in a closed state, and to open based on a control signal sent from the controller 97 when the measured value of the second pressure sensor 95B exceeds the predetermined threshold value.

The opening of the relief valve for the abdominal cavity side and/or that of the relief valve for the lumen side allow carbon dioxide gas in the abdominal cavity AC and/or the lumen BC to be released, thereby reducing a pressure inside the abdominal cavity AC and/or the lumen BC.

The system controller 5 is provided with an image processing unit P1 operative to execute image processing related to the abdominal cavity image and/or the lumen image based on a mode signal sent from the controller 97 of the gas supply apparatus 41.

FIG. 6 is a block diagram illustrating a functional structure of the image processing unit P1.

As illustrated in FIG. 6, the system controller 5 is electrically connected to the first CCU 23, the second CCU 33, the gas supply apparatus 41, and the monitor 6 set forth above.

Specifically, the system controller 5 has a function of receiving the first and second image signals sent from the first and second CCUs 23 and 33, and of generating the abdominal cavity image and the lumen image based on the first and second image signals. The system controller 5 also has a function of superimposing the generated abdominal cavity image and the lumen image to generate a composite image, thereby sending the composite image to the monitor 6.

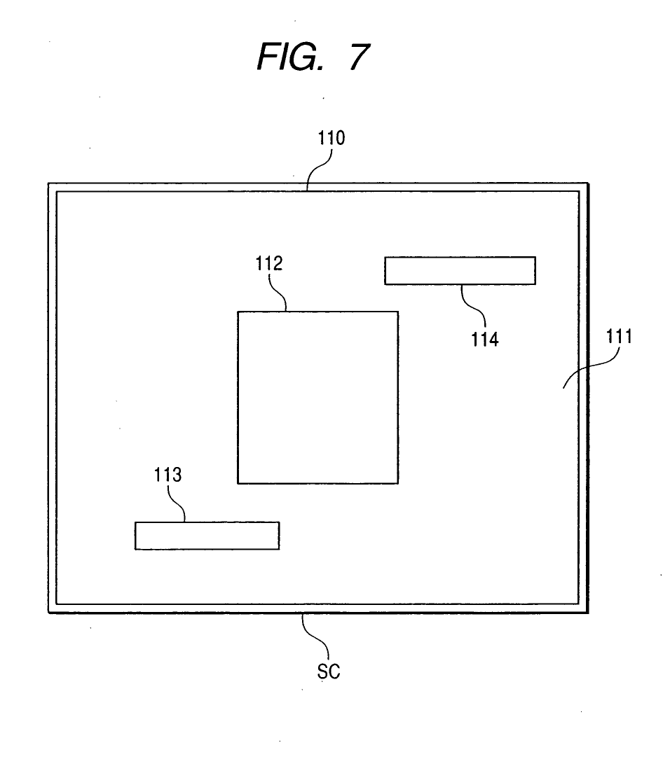

FIG. 7 illustrates an example of the composite image 110 displayed on a screen SC of the monitor 6.

As illustrated in FIG. 7, the composite image 110 consists of a main image 111, a sub-image 112, and text images 113 and 114. The text images 113 and 114 are related to the abdominal cavity AC and the lumen BC, respectively. The position and the scale of the sub-image 112 with respect to the main image 111 are previously determined, and the text images 113 and 114 are designed to be displayed on predetermined positions in the main image 111, respectively.

As illustrated in FIG. 6, the image processing unit P1 of the system controller 5 functionally has a text image generating module 101, an image composition module 102, a video signal processing module 103, a determining module 104, and a control module 105.

Incidentally, the system controller 5 may has at least one CPU and at least one memory connected to the at least one CPU. The modules 101 to 105 can be configured as operations (functions) of the at least one CPU based on program(s) stored in the at least one memory. Moreover, the image processing unit P1 itself can be configured as a dedicated computer circuit having at least one CPU and at least one memory connected to the at least one CPU and operative to execute image processing only. Furthermore, each of the functional blocks 101 to 105 can be configured as a computer circuit having at least one CPU and at least one memory connected to the at least one CPU.

The text image generating module 101 has a function of generating text images based on textual information related to the abdominal cavity AC and the lumen BC.

The image composition module 102 has a first function of generating the composite image based on the abdominal-cavity image corresponding to the first image signal sent from the first CCU 23 and the lumen image corresponding to the second image signal sent from the second CCU 33 based on control of the control module 105 to generate the composite image.

Specifically, the image composition module 102, as the first function, generates one of the abdominal-cavity image and the lumen image as the main image 111 based on an image-selection control signal CS1 sent from of the control module 105. Subsequently, the image composition module 102, as the first function, subjects the other image to image processing to superimpose it on the main image 111 as the sub-image 112 such that the sub-image 112 is positioned at a predetermined position on the main image 111 with a predetermined scale with respect thereto based on an image-superimposing control signal CS2 sent from the control module 105. This image processing generates the composite image.

In addition, the image composition module 102 has a second function of superimposing the text images generated by the text image generating module 101 on the composite image at predetermined positions thereof based on a text image control signal CS3 sent from the control module 105.

The video signal processing module 103 has a function of converting the composite image into a standard video signal, such as PAL (Phase Alternating Line) signal, or an NTSC (National Television System Committee) signal.

The determining module 104 has a function of determining whether the carbon dioxide gas is supplied through the first CO₂ supply path DC1 or the second CO₂ supply path DC2 based on a mode signal provided from the controller 97 of the gas supply apparatus 41.

Specifically, the determining module 104 determines, based on the mode signal, whether the gas supply apparatus 41 operates in the abdominal-cavity insufflation mode to insufflate the carbon dioxide gas into the abdominal cavity AC or in the lumen insufflation mode to insufflate the carbon dioxide gas into the lumen BC. The determining module 104, as the function, sends the determined result indicative of the abdominal-cavity insufflation mode or the lumen insufflation mode to the control module 105.

The control module 105 has a function of providing the control signals CS1 to CS3 to the image composition module 102.

Incidentally, as illustrated in FIG. 6, an input unit 106 having a pointing device including keyboard and/or a mouse pointer, which allows an operator to specify positions on the screen SC of the monitor 6 and enter items of information thereon, can be connected to the control module 105.

Next, operations of the surgical system 1 according to the first embodiment will be described hereinafter.

For example, when performing laparoscopic surgery employing the surgical system 1, the operator inserts the rigidscope 21 into the inside of the abdominal cavity AC with the flexiblescope 31 being inserted into the lumen BC, such as a large intestine present in the abdominal cavity AC. The operator specifies and treats at least one site to be treated in the abdominal cavity AC and/or the lumen BC.

Next, the power switch 71 is turned on by, for example, the operator. In response to the turning-on of the switch 71, the pressure display 77b of the front panel FP is ready to display the measured value by the first pressure sensor 95A, and the pressure display 81a of the front panel FP is ready to display the measured value by the second pressure sensor 95B. In addition, the foot switch 44 becomes a state that allows the operator to operate it.

On the pressure display 77b, the pressure setting inside the abdominal cavity AC, which has been previously set on, for example, the center operation panel 8, is displayed. Similarly, on the flow-rate display 78b, the flow-rate setting of the carbon dioxide gas to be insufflated into the abdominal cavity AC, which has been previously set on, for example, the center operation panel 8, is displayed.

On the pressure display 81b, the pressure setting of the carbon dioxide gas to be insufflated into the lumen BC, which has been previously set on, for example, the center operation panel 8, is displayed. Similarly, on the flow-rate display 80b, the flow-rate setting of the carbon dioxide gas to be insufflated into the lumen BC, which has been previously set on, for example, the center operation panel 8, is displayed.

In cases where no pressure setting and/or flow-rate setting inside the abdominal cavity AC have been previously determined, the operator appropriately can operate the pressure setting buttons 74a and 74b and/or the flow-rate setting buttons 75a and 75b to determine the pressure setting and/or flow-rate setting inside the abdominal cavity AC. The instruction corresponding to the pressure setting and/or flow-rate setting inside the abdominal cavity AC is sent from the manually operable setting section 63 to the controller 97. Similarly, in cases where no pressure setting and/or flow-rate setting inside the lumen BC have been previously determined, the operator appropriately can operate the pressure setting buttons 86a and 86b and/or the flow-rate setting buttons 85a and 85b to determine the pressure setting and/or flow-rate setting inside the lumen BC. The instruction corresponding to the pressure setting and/or flow-rate setting inside the lumen BC is sent from the manually operable setting section 63 to the controller 97.

In response to operations of the abdominal cavity select button 82 and the gas-supply start button 72, the gas supply apparatus 41 starts to insufflate the carbon dioxide gas into the abdominal cavity AC with its pressure regulated suitable for the abdominal cavity AC. Specifically, the controller 97 continuously controls the opening of the first electropneumatic proportional valve 93A, so that the pressure and the flow-rate inside the abdominal cavity AC are maintained approximately to the pressure setting and flow-rate setting established on the font panel FP, respectively.

On the other hand, in response to operations of the lumen select button 83 and the switch portion 44a of the foot switch 44 starts to insufflate the carbon dioxide gas into the lumen BC with its pressure regulated suitable for the lumen BC. Specifically, the controller 97 continuously controls the opening of the second electropneumatic proportional valve 93B, so that the pressure and the flow-rate inside the lumen are maintained approximately to the pressure setting and flow-rate setting established on the font panel FP.

Specifically, when the cock of the CO₂ bottle 42 is opened by the operator or an assistant, the opening of the cock of the CO₂ bottle 42 causes the carbon dioxide gas to flow out of the bottle 42 through the high-pressure gas tube 46, thereby flowing into the gas supply apparatus 41. The gas flowing into the apparatus 41 is introduced through the first delivery channel C1 to the pressure reducing unit 92.

When the abdominal cavity select button 82 is turned on, the controller 97 enters the abdominal-cavity insufflation mode.

In the abdominal-cavity insufflation mode, the controller 97 sends the control signals to the first electropneumatic proportional valve 93A and the first solenoid valve 93A, respectively. The control signal sent to the first electropneumatic proportional valve 93A allows it to open by a predetermined opening corresponding to the appropriate insufflation pressure range of 0 to 80 mmHg or thereabout and the appropriate insufflation flow-rate range of 0.1 to 35 L/min or thereabout. The control signal sent to the first solenoid valve 94A allows it to open. In this case, the second electropneumatic proportional valve 93B and the second solenoid valve 94B are kept closed.

As a result, the carbon dioxide gas supplied up to the inlet of the first electropneumatic proportional valve 93A flows through the first electropneumatic proportional valve 93A so that the pressure and the flow-rate of the carbon dioxide gas are regulated within the corresponding predetermined ranges suitable for the insufflation of the abdominal cavity AC, respectively. The carbon dioxide gas with its pressure and flow-rate being regulated, respectively, is introduced into the first CO₂ supply path DC1.

In the abdominal-cavity insufflation mode, because the second electropneumatic proportional valve 93B and the second solenoid valve 94B are kept closed, no carbon dioxide gas is supplied into the second CO₂ supply path DC2. The carbon dioxide gas therefore passes through the first CO₂ supply path DC1, that is, the first solenoid valve 94A, the first flow-rate sensor 96A, the first adapter 41A, the abdominal cavity tube 45a, and the third trocar 16 to be insufflated into the abdominal cavity AC.

While the carbon dioxide gas is supplied into the abdominal cavity AC, the first pressure sensor 95A measures the pressure of the carbon dioxide gas flowing through the first CO₂ supply path DC1, and the first flow-rate sensor 96A measures the flow rate of the carbon dioxide gas flowing through the first CO₂ supply path DC1. The first pressure sensor 95A and the first flow-rate sensor 96A send the measured results to the controller 97.

The controller 97 receives the measured results. The controller 97 controls the opening of the first electropneumatic proportional valve 93A based on the measured results. The control of the opening of the valve 93A causes the pressure and the flow-rate of the carbon dioxide gas into the abdominal cavity AC to be regulated within the corresponding range of, for example, 0 to 80 mmHg or thereabout and that of, for example, 0.1 to 35 L/min thereabout, respectively.

Incidentally, the controller 97 of the gas supply apparatus 41 is configured to measure the pressure inside the abdominal cavity AC with the first solenoid valve 94A closed.

In addition, in the first embodiment, the controller 97 sends the mode signal indicative of the abdominal-cavity insufflation mode to the system controller 5.

When the lumen select button 83 is turned on or the switch portion 44a of the foot switch 44 is turned on, the controller 97 enters the lumen insufflation mode.

In the lumen insufflation mode, the controller 97 sends the control signals to the second electropneumatic proportional valve 93B and the second solenoid valve 93B, respectively. The control signal sent to the second electropneumatic proportional valve 93B allows it to open by a predetermined opening corresponding to the appropriate insufflation pressure range of 0 to 500 mmHg or thereabout and the appropriate insufflation flow-rate range of 1 to 3 L/min or thereabout. The control signal sent to the second solenoid valve 94B allows it to open. In this case, the first electropneumatic proportional valve 93A and the first solenoid valve 94A are kept closed.

As a result, the carbon dioxide gas supplied up to the inlet of the second electropneumatic proportional valve 93B flows through the second electropneumatic proportional valve 93B so that the pressure and the flow-rate of the carbon dioxide gas are regulated within the corresponding predetermined ranges suitable for the insufflation of the lumen BC, respectively. The carbon dioxide gas with its pressure and flow-rate being regulated, respectively, is introduced into the second CO₂ supply path DC2.

In the lumen insufflation mode, because the first electropneumatic proportional valve 93A and the first solenoid valve 94A are kept closed, no carbon dioxide gas is supplied into the first CO₂ supply path DC1. The carbon dioxide gas therefore passes through the second CO₂ supply path DC2, that is, the second solenoid valve 94B, the second flow-rate sensor 96B, the second adapter 41B, the lumen tube 45b, the adapter 43, and the gas delivery channel SC inside the flexiblescope 31 to be insufflated into the lumen BC.

While the carbon dioxide gas is supplied into the lumen BC, the second pressure sensor 95B measures the pressure of the carbon dioxide gas flowing through the second CO₂ supply path DC2, and the second flow-rate sensor 96B measures the flow rate of the carbon dioxide gas flowing through the second CO₂ supply path DC2. The second pressure sensor 95B and the second flow-rate sensor 96B send the measured results to the controller 97.

The controller 97 receives the measured results. The controller 97 controls the opening of the second electropneumatic proportional valve 93B based on the measured results. The control of the opening of the valve 93B causes the pressure and the flow-rate of the carbon dioxide gas into the lumen BC to be regulated within the corresponding range of, for example, 0 to 500 mmHg or thereabout and that of, for example, 1 to 3 L/min thereabout, respectively.

Incidentally, the controller 97 of the gas supply apparatus 41 is configured to measure the pressure inside the lumen BC with the second solenoid valve 94B closed.

In addition, in the first embodiment, the controller 97 sends the mode signal indicative of the lumen insufflation mode to the system controller 5.

As set forth above, the abdominal-cavity pressure control operations allow the pressure inside of the abdominal cavity AC to be kept to the pressure setting or thereabout, which has been set by the operator. Similarly, the lumen-pressure control operations allow the pressure inside of the lumen BC to be kept to the pressure setting or thereabout, which has been set by the operator.

Under such a state, an optical image of a target, such as the site to be treated in the abdominal cavity AC is captured by the rigidscope 21, and the captured image (rigidscope image) is picked up by the image pickup device 24a of the camera 24. The pickup device 24a sends the picked up image to the first CCU 23 as the first image signal.

The first CCU 23 receives the first image signal to subject the received first image signal to image processing of necessity. The first CCU 23 outputs the image-processed first image signal to the image processing unit P1.

On the other hand, an optical image of a target, such as the site to be treated in the lumen BC is captured by the flexiblescope 31, and the captured image (flexiblescope image) is picked up by the image pickup device 31a of the flexiblescope 31. The pickup device 31a sends the picked up image to the second CCU 33 as the second image signal.

The second CCU 33 receives the second image signal to subject the received second image signal to image processing of necessity. The second CCU 33 outputs the image-processed second image signal to the image processing unit P1.

The controller 97 of the gas supply apparatus 41 sends the textual information related to the abdominal cavity AC and/or the lumen BC to the image processing unit P1.

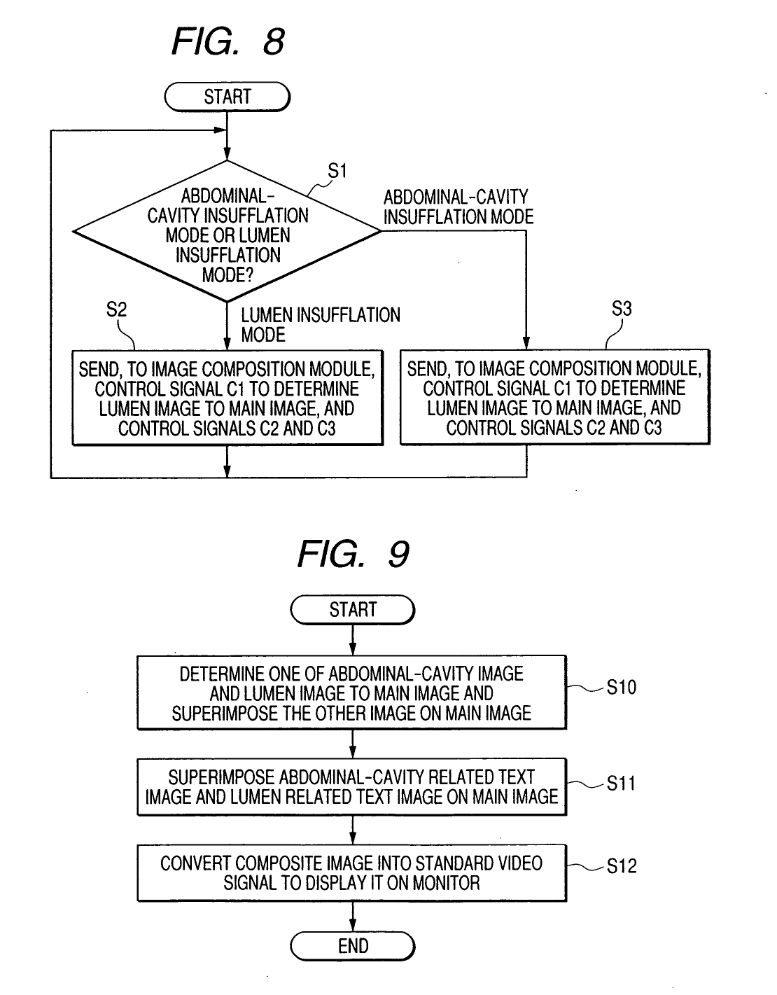

The image processing unit P1 executes the following operations illustrated in FIG. 8.

Specifically, the determining module 104 of the image processing unit P1 receives the mode signal sent from the controller 97 to determine whether the operation mode of the gas supply apparatus 41 is the abdominal-cavity insufflation mode or the lumen insufflation mode (FIG. 8; step S1).

The determining module 104 sends the determined result to the control module 105.

When the gas supply apparatus 41 currently operates in the lumen insufflation mode, the determined result indicates the “lumen insufflation mode”, so that, in step S2, the control module 105 sends the image-selection control signal CS1 representing that the lumen image is determined to the main image 111 to the image composition module 102. In addition, in step S2, the control module 105 sends, to the image composition module 102, the image-superimposing control signal CS2 representing that the abdominal cavity image as the sub-image 112 is superimposed on the main image 111 at a predetermined position thereof with a predetermined scale.

In step S2, the control module 105 sends the text image control signal CS3 representing that the text images generated by the text image generating module 101 are superimposed on the main image 111 at predetermined positions thereof to the image composition module 102.

When the gas supply apparatus 41 currently operates in the abdominal-cavity insufflation mode, the determined result indicates the “abdominal-cavity insufflation mode”, so that, in step S3, the control module 105 sends the image-selection control signal CS1 representing that the abdominal cavity image is determined to the main image 111 to the image composition module 102. In addition, in step S3, the control module 105 sends, to the image composition module 102, the image-superimposing control signal CS2 representing that the lumen image as the sub-image 112 is superimposed on the main image 111 at a predetermined position thereof with a predetermined scale.

In step S3, the control module 105 sends the text image control signal CS3 representing that the text images generated by the text image generating module 101 are superimposed on the main image 111 at predetermined positions thereof to the image composition module 102.

On the other hand, the text image generating module 101 generates an abdominal-cavity relevant text image based on the textual information concerning the abdominal cavity AC, and a lumen relevant text image based on the textual information concerning the lumen BC. The text image generating module 101 sends the generated abdominal-cavity relevant text image and the lumen relevant text image to the image composition module 102.

The image composition module 102 executes the operations illustrated in FIG. 9 based on the control signals CS1-CS3 sent from the control module 105, the first and second image signals sent from the first and second CCU 23 and 33, the abdominal cavity relevant and lumen relevant text images sent from the text image generating module 101.

Specifically, in step S10, the image composition module 102 generates the abdominal-cavity image and the lumen image based on the first and second image signals, respectively. In step S10, the image composition module 102 selects one of the generated abdominal-cavity image and the lumen image as the main image 111 based on the image-selection control signal CS1. Subsequently, in step S10, the image composition module 102 reduces the other of the abdominal-cavity image and the lumen image by the predetermined scale included in the image-superimposing control signal CS2. Next, in step S10, the image composition module 102 superimposes the reduced image with the predetermined scale on a predetermined position (pixel area) of the main image 111 as the sub-image 112; this predetermined position is included in the control signal CS2.

In step S11, the image composition module 102 superimposes the abdominal-cavity relevant text image on a first predetermined position (pixel area) of the main image 111, and the lumen relevant text image on a second predetermined position (pixel area) thereof; these first and second predetermined positions are included in the text image control signal CS3.

These operations by the image composition module 102 illustrated in FIG. 9 provide the composite image 110 consisting of the main image 111, the sub-image 112, the abdominal-cavity relevant text image 113, and the lumen relevant text image 114. The image composition module 102 sends the composite image 110 generated based on the operations in steps S10 and S11 to the video signal processing module 103.

The video signal processing module 103 receives the composite image 110 sent from the image composition module 102 to convert the received composite image 110 into the standard video signal, thereby displaying the standard video signal on the screen SC of the monitor 6 in step S12.

The determining module 104 and the control module 105 of the image processing unit P1 repeatedly execute the operations in steps S1 to S3 until the laparoscopic surgery is completed. In response to the operations in steps S1 to S3, the image composition module 102 and the video signal processing module 103 of the image processing unit P1 repeatedly execute the operations in steps S10 to S12.

These operations of the image processing unit P1 allow switching of the main image 111 between the abdominal-cavity image and the lumen image based on the current operation mode of the gas supply apparatus 41.

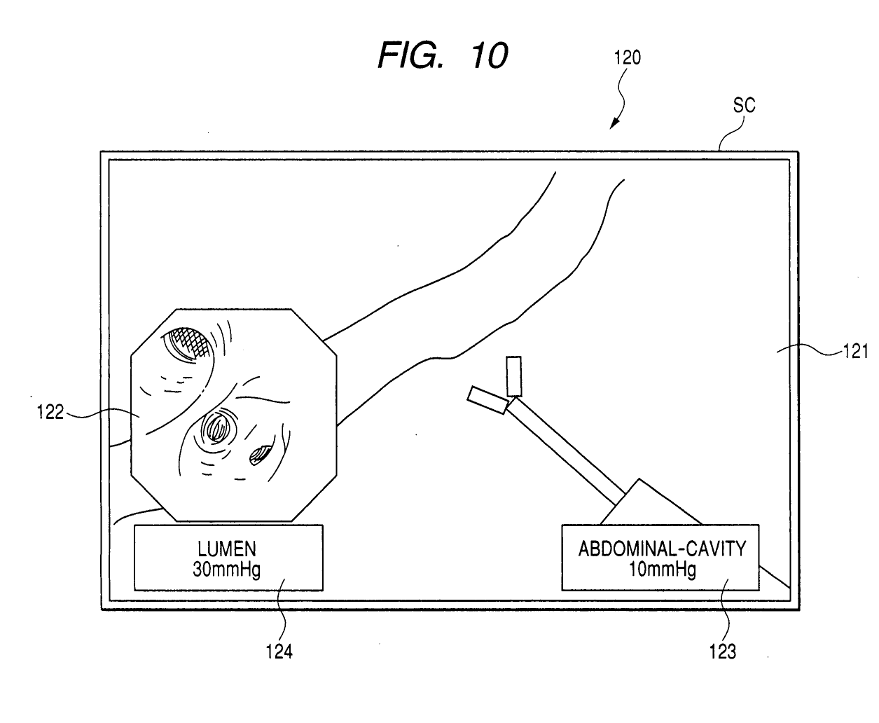

For example, composite images 120, 130, and 140 can be displayed on the screen SC of the monitor 6 illustrated in FIGS. 10 to 12, respectively.

In the composite image 120 illustrated in FIG. 10, the abdominal-cavity image 121 is displayed on the screen SC as the main image, and the lumen image 122 is superimposed on the abdominal-cavity image 121 when the gas supply apparatus 41 operates in the abdominal cavity insufflation mode.

In addition, at predetermined positions on the main image 121, the abdominal-cavity relevant text image and the lumen relevant text image are displayed. In FIG. 10, the abdominal-cavity relevant text image 123 indicative of the pressure value inside the abdominal cavity AC and measured by the first pressure sensor 95A is displayed. Moreover, in FIG. 10, the lumen relevant text image 124 indicative of the pressure value inside the lumen BC and measured by the second pressure sensor 95B is displayed.

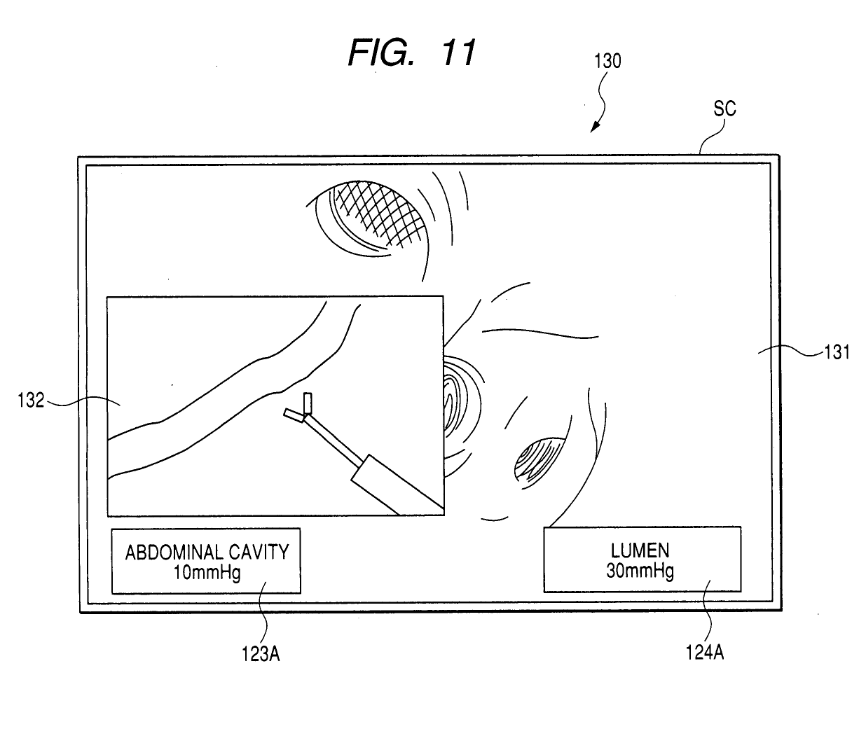

On the other hand, in the composite image 130 illustrated in FIG. 11, the lumen image 131 is displayed on the screen SC as the main image, and the abdominal-cavity image 132 is superimposed on the lumen image 131 when the gas supply apparatus 41 operates in the lumen insufflation mode.

In addition, at predetermined positions on the main image 131, the abdominal-cavity relevant text image and the lumen relevant text image are displayed, which is similar to the composite image 120. In FIG. 11, the abdominal-cavity relevant text image 123A indicative of the pressure value inside the abdominal cavity AC and measured by the first pressure sensor 95A is displayed. Moreover, in FIG. 11, the lumen relevant text image 124A indicative of the pressure value inside the lumen BC and measured by the second pressure sensor 95B is displayed.

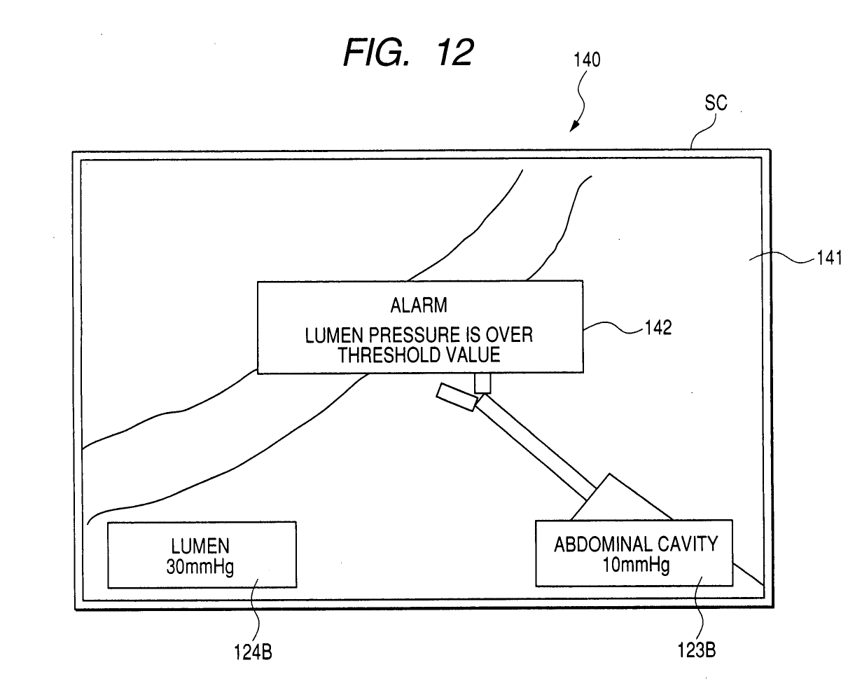

In the composite image 140 illustrated in FIG. 12, the abdominal-cavity image 141 is displayed on the screen SC as the main image when the gas supply apparatus 41 operates in the abdominal-cavity insufflation mode, and no sub-image (lumen image) is displayed thereon.

At predetermined positions on the main image 141, as illustrated in FIG. 12, the abdominal-cavity relevant text image 123B indicative of the pressure value inside the abdominal cavity AC and measured by the first pressure sensor 95A is displayed. Moreover, in FIG. 12, the lumen relevant text image 124B indicative of the pressure value inside the lumen BC and measured by the second pressure sensor 95B is displayed.

In addition to the text images 123B and 124B, a text image 142 representing that the pressure inside the lumen BC exceeds the threshold value is displayed on the main image 141. Textual information corresponding to the text image 142 is sent from the gas supply apparatus 41 so that the text image 142 is superimposed on the main image 141 based on the operations of the text image generating module 101, the image composition module 102, and the control module 105.

Note that main images and sub-images displayed on the screen SC of the monitor 6 are not limited to the positions and sizes illustrated in FIGS. 10 to 12. In addition, for example, a main image and a sub-image do not separate the screen SC of the monitor 5.

That is, display positions of a main image, a sub-image, and text images related to the abdominal cavity AC and the lumen BC on the screen SC and sizes thereof can be determined according to the operator's will.

As described above, in the surgery system 1 according to the first embodiment, both the abdominal-cavity image and the lumen image can be displayed on the single monitor 6. This permits the operator manipulating the rigidscope 21 and that manipulating the flexiblescope 31 to perform the laparoscopic surgery while monitoring the abdominal-cavity image and the lumen image displayed on the monitor 6, respectively.

This allows each operator to visibly recognize both the abdominal-cavity image and the lumen image concentrately at once, making it possible for each operator to accurately rapidly grasp the at least one site(s) to be treated in the abdominal cavity AC and/or the lumen BC.

Especially, in the first embodiment, switching of the operation mode of the gas supply apparatus 41 allows the main image to be automatically switched between the abdominal-cavity image and the lumen image. This makes it possible for each operator to easily grasp condition inside one of the abdominal cavity and the lumen into which the carbon dioxide gas is currently being insufflated.

In addition, the surgery system 1 according to the first embodiment allows both the abdominal-cavity image and lumen image to be displayed without using two monitors, one of which displays the abdominal-cavity image and the other thereof displays the lumen image, making it possible to downscale the whole of the surgery system 1.

In the first embodiment, the gas supply apparatus 41 is configured to supply the carbon dioxide gas as the predetermined gas, but the present invention is not limited to the structure. Specifically, the gas supply apparatus 41 can be configured to supply inactive gas, such as helium gas, as the predetermined gas.

When the input unit 106 is connected to the control module 105 (see FIG. 6), the input unit 106 allows the operators to enter composite image positional information into the control module 105. The composite image positional information represents the position (pixel area) and the size of the sub-image on the main image, and/or the positions (pixel areas) of the text images. In this case, the image composition module 102 determines the sizes of the main image, sub-image, and the text images, respectively, and the positional relationship therebetween based on the entered composite image positional information. Thereafter, the image composition module 102 superimposes the sub-image and the text images on the main image based on the determined sizes and positional relationships (see FIG. 9 steps S10 to S11).

When the gas supply apparatus 41 currently operates in one of the abdominal-cavity insufflation mode and the lumen insufflation mode, the surgery system 1 according to the first embodiment can display corresponding one of the abdominal-cavity image and the lumen image on the whole of the screen SC of the monitor 6.

In this case, the surgery system 1 can switch one of the abdominal-cavity image and the lumen image being displayed on the whole of the screen SC of the monitor 6 to the other thereof in response to switching operation of the current operation mode of the gas supply apparatus 411.

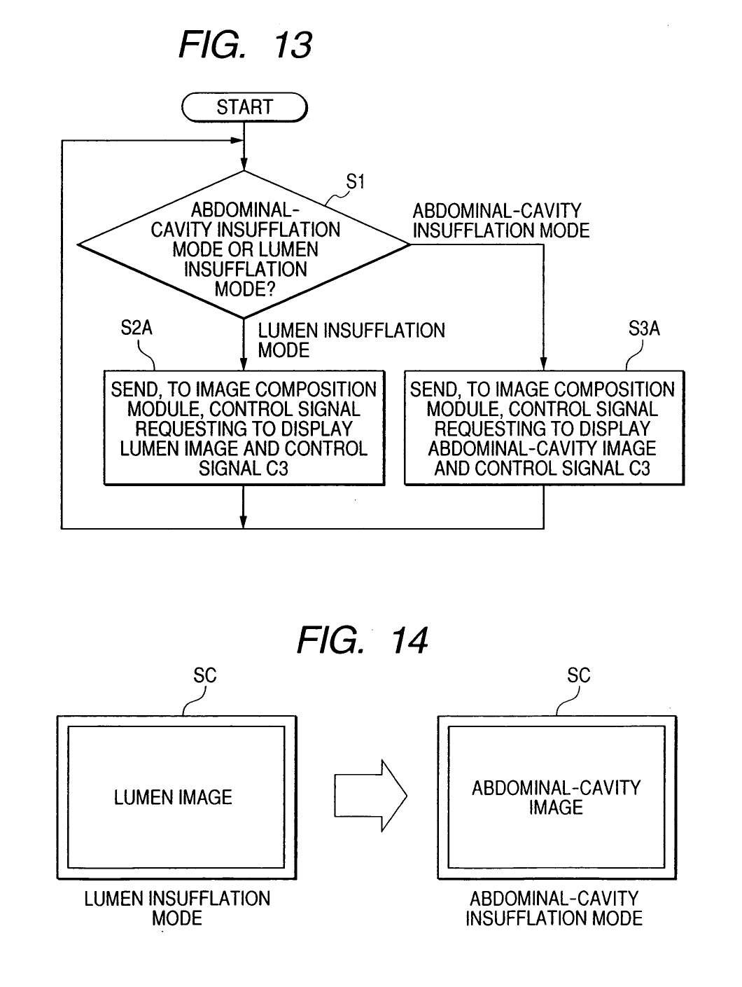

For example, as illustrated in FIG. 13, when the determined result in step S1 indicates the “lumen insufflation mode”, the control module 105 sends a control signal requesting to display the lumen image on the monitor 6 together with the control signal CS3 to the image composition module 102 (step S2A).

When the determined result in step S1 indicates the “abdominal-cavity insufflation mode”, the control module 105 sends a control signal requesting to display the abdominal-cavity image on the monitor 6 together with the control signal CS3 to the image composition module 102 (step S3A).

As a result, the operations of the modules 102 and 103 in steps S10 to S12 allow automatic switching from the lumen image displayed on the whole of the screen SC of the monitor 6 to the abdominal-cavity image when the determined result is switched from the “lumen insufflation mode” to “abdominal-cavity insufflation mode” (see FIG. 14).

That is, the surgery system according to the modification of the first embodiment allows automatic switching from one of the abdominal-cavity image and the lumen image, which is displayed on the monitor 6, to the other thereof depending on the current operation mode of the gas supply apparatus 41 without generating composite images.

This results in that, as in the first embodiment, it is possible to easily grasp condition inside one of the abdominal cavity and the lumen into which the carbon dioxide gas is currently being insufflated.

### Second Embodiment

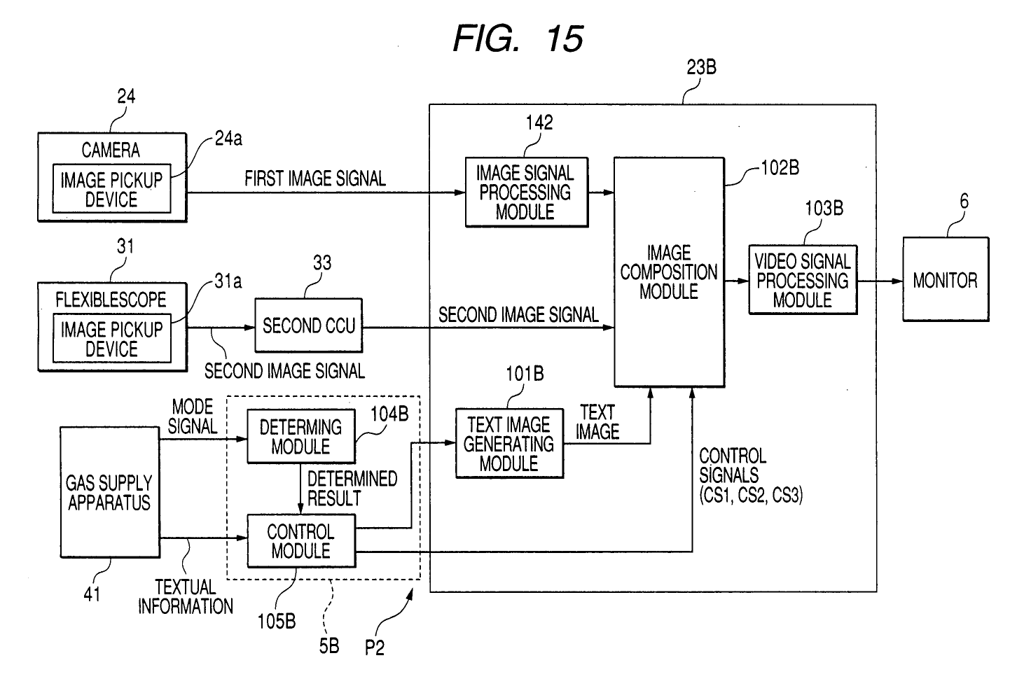

FIG. 15 is a block diagram illustrating a functional structure of an image processing unit P2 according to a second embodiment of the present invention.

In the first embodiment, the elements 101 to 105 constituting the image processing unit P1 are configured to functions of the system controller 5. In the second embodiment, however, elements corresponding to the determining module and control module are configured to functions of the system controller, and the remaining elements corresponding to the text image generating module, image composition module, and the video signal processing module) are configured to functions of the first CCU. Because other elements of the surgical system according to the second embodiment are substantially identical with those of the surgical system according to the first embodiment, the descriptions of which are omitted.

Reference characters and/or numerals assigned to elements of the surgical system according to the second embodiment, which are substantially identical to those of the surgical system 1, are the same as those assigned to the elements of the surgical system 1.

As illustrated in FIG. 15, in the surgical system 1B according to the second embodiment, the image processing unit P2 includes a text image generating module 101B, an image composite module 102B, and a video signal processing module 103B, which are substantially identical with the modules 101, 102, and 103, respectively. The modules 101B to 103B are installed in a first CCU 23B.

The first CCU 23B includes an image signal processing module 142 operative to subject the first image signal sent from the camera 24 to image processing of necessity to convert it into a signal that is processable by the image composition module 102B, thereby outputting the converted first image signal thereto.

In addition, the image processing unit P2 includes a determining module 104B and a control module 105B, which are installed in a system controller 5B.

The text image generating module 101B, as in the first embodiment, has a function of generating the text images based on the textual information related to the abdominal cavity AC and the lumen BC.

The image composition module 102B has a first function of generating the composite image based on the abdominal-cavity image corresponding to the first image signal outputted from the module 142 and the lumen image corresponding to the second image signal outputted from the second CCU 33 based on control of the control module 105 to generate the composite image.

Specifically, the image composition module 102B, as the first function, generates one of the abdominal-cavity image and the lumen image as the main image 111 based on the image-selection control signal CS1 sent from of the control module 105. Subsequently, the image composition module 102B, as the first function, subjects the other image to image processing to superimpose it on the main image 111 as the sub-image 112 such that the sub-image 112 is positioned at a predetermined position on the main image 111 with a predetermined scale with respect thereto based on the image-superimposing control signal CS2 sent from the control module 105. This image processing generates the composite image.

In addition, the image composition module 102B has a second function of superimposing the text images generated by the text image generating module 101B on the composite image at predetermined positions thereof based on the text image control signal CS3 sent from the control module 105B.

The determining module 104B of the system controller 5B has a function of determining whether the carbon dioxide gas is supplied through the first CO₂ supply path DC1 or the second CO₂ supply path DC2 based on a mode signal provided from the controller 97 of the gas supply apparatus 41.

Specifically, the determining module 104B determines, based on the mode signal, whether the gas supply apparatus 41 operates in the abdominal-cavity insufflation mode to insufflate the carbon dioxide gas into the abdominal cavity AC or in the lumen insufflation mode to insufflate the carbon dioxide gas into the lumen BC. The determining module 104B, as the function, sends the determined result indicative of the abdominal-cavity insufflation mode or the lumen insufflation mode to the control module 105B.

The control module 105B of the system controller 5B has a function of providing the control signals CS1 to CS3 to the image composition module 102B.

Incidentally, as in the first embodiment, an input unit having a pointing device including keyboard and/or a mouse pointer, which allows an operator to specify positions on the screen SC of the monitor 6 and enter items of information thereon, can be connected to the control module 105B.

In the surgery system 1B according to the second embodiment, the control module 105B of the system controller 5B executes the operations in steps S1 to S3 of FIG. 8. In addition, the image composition module 102B and the video signal processing module 103B of the first CCU 23B execute the operations in steps S10 to S12. These operations allow the surgery system 1B to obtain the effects that are the substantially same as those obtained by the surgery system 1 according to the first embodiment.

Incidentally, in the second embodiment, the text image generating module 101B, the image composition module 102B, and the video signal processing module 103B provided in the first CCU 23B allow the first CCU 23B to combine the abdominal-cavity image based on the first image signal and the lumen image based on the second image signal sent from the second CCU 33. In addition, the modules 101B to 103B allow the first CCU 23B to switchably display one of the abdominal-cavity image and the lumen image on the monitor 6. The present invention is however limited to the structure.

Specifically, providing the text image generating module 101B, the image composition module 102B, and the video signal processing module 103B in the second CCU 33 allow the second CCU 33 to combine the abdominal-cavity image based on the first image signal sent from the first CCU 23 and the lumen image based on the second image signal. In addition, the modules 101B to 103B allow the second CCU 33 to switchably display one of the abdominal-cavity image and the lumen image on the monitor 6.

### Third Embodiment

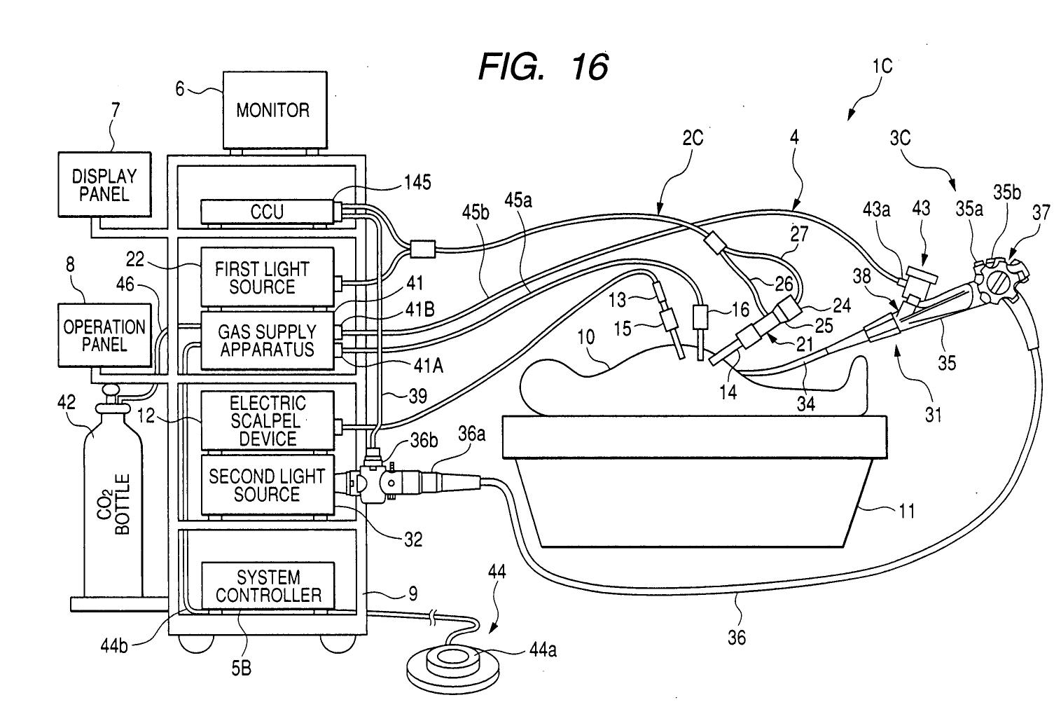

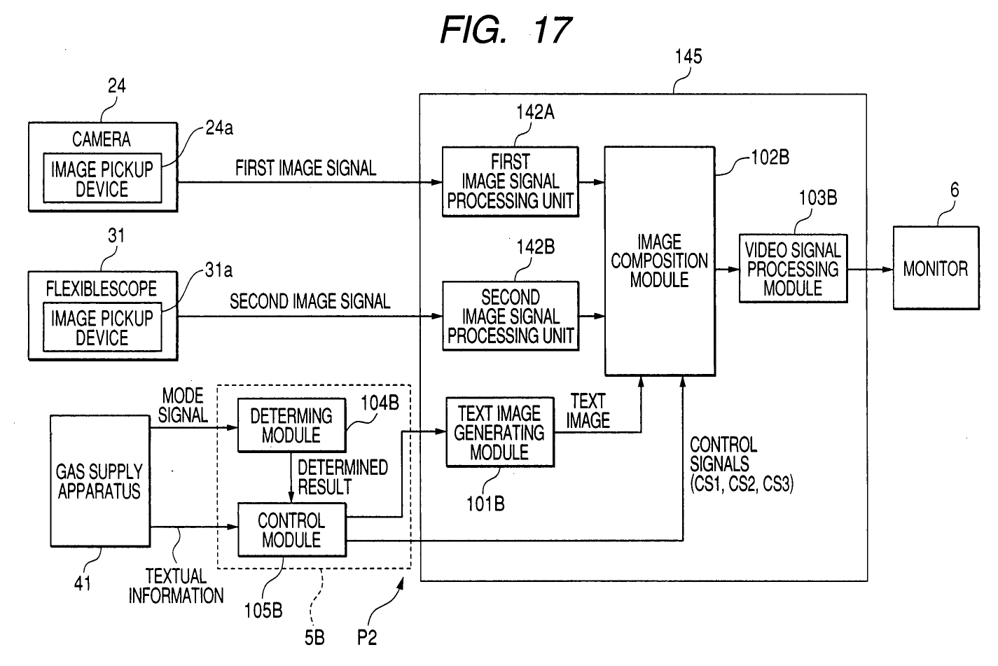

FIG. 16 is an overall structural view schematically illustrating the structure of a surgical system according to a third embodiment of the present invention, and FIG. 17 is a block diagram illustrating a functional structure of an image processing unit P3 according to the third embodiment of the present invention.

In the third embodiment, CCUs are not individually prepared for the rigidscope 21 and the flexiblescope 31, but a single CCU is prepared to serve as a CCU for the rigidscope 21 and that for the flexiblescope 31.

In addition, as in the second embodiment, elements of an image processing unit according to the third embodiment, which correspond to the determining module and control module, are configured to functions of the system controller. The remaining elements corresponding to the text image generating module, image composition module, and the video signal processing module) are configured to functions of the single CCU. Because other elements of the surgical system according to the third embodiment are substantially identical with those of the surgical system according to the second embodiment, the descriptions of which are omitted.

Reference characters and/or numerals assigned to elements of the surgical system according to the third embodiment, which are substantially identical to those of the surgical system 1B, are the same as those assigned to the elements of the surgical system 1B.

As illustrated in FIG. 16, a surgery system 1C according to the third embodiment is provided with the single CCU 145 in place of the first CCU 23 and those of the second CCU 33; this CCU 145 is operative to execute the functions of the first CCU 23 and those of the second CCU 33.

Specifically, a first endoscope system 2C has the CCU 145 in place of the first CCU 23, and a second endoscope system 3C has the CCU 145 in place of the second CCU 33.

The CCU 145 is electrically connected to the camera 24 through the image pickup cable 27 such that the first image signal sent from the image pickup device 24a of the camera 24 enters into the CCU 145 through the image pickup cable 27. In addition, the CCU 145 is electrically connected to the flexiblescope 31 through the electric cable 39 such that the second image signal sent from the image pickup device 31a of the flexiblescope 31 enters into the CCU 145 through the electric cable 39.

Next, a functional structure of the CCU 145 will be described hereinafter.

The CCU 145, as illustrated in FIG. 17, includes a text image generating module 101C, an image composite module 102C, and a video signal processing module 103C as elements of the image processing unit P3.

The text image generating module 101C, image composite module 102C, and video signal processing module 103C are substantially identical with the modules 101B, 102B, and 103B described in the second embodiment.

The CCU 145 includes a first image signal processing module 142A operative to subject the first image signal sent from the camera 24 through the image pickup cable 27 to image processing of necessity to convert it into a signal that is processable by the image composition module 102C, thereby outputting the converted first image signal thereto.

In addition, the CCU 145 includes a second image signal processing module 142B operative to subject the second image signal sent from the image pickup device 31a through the electric cable 39 to image processing of necessity to convert it into a signal that is processable by the image composition module 102C, thereby outputting the converted second image signal thereto.

The image processing unit P3 includes the determining module 104B and the control module 105B installed in the system controller 5B.

The text image generating module 101C, as in the first and second embodiments, has a function of generating the text images based on the textual information related to the abdominal cavity AC and the lumen BC.

The image composition module 102C has a first function of generating the composite image based on the abdominal-cavity image corresponding to the first image signal outputted from the module 142A and the lumen image corresponding to the second image signal outputted from the module 142B based on control of the control module 105C to generate the composite image.

Specifically, the image composition module 102C, as the first function, generates one of the abdominal-cavity image and the lumen image as the main image 111 based on the image-selection control signal CS1 sent from of the control module 105C. Subsequently, the image composition module 102C, as the first function, subjects the other image to image processing to superimpose it on the main image 111 as the sub-image 112 such that the sub-image 112 is positioned at a predetermined position on the main image 111 with a predetermined scale with respect thereto based on the image-superimposing control signal CS2 sent from the control module 105C. This image processing generates the composite image.

In addition, the image composition module 102C has a second function of superimposing the text images generated by the text image generating module 101C on the composite image at predetermined positions thereof based on the text image control signal CS3 sent from the control module 105C.

The control module 105B of the system controller 5B has a function of providing the control signals CS1 to CS3 to the image composition module 102C.

Incidentally, as in the first and second embodiments, an input unit having a pointing device including keyboard and/or a mouse pointer, which allows an operator to specify positions on the screen SC of the monitor 6 and enter items of information thereon, can be connected to the control module 105B.

In the surgery system 1C according to the third embodiment, the control module 105B of the system controller 5B executes the operations in steps S1 to S3 of FIG. 8. In addition, the image composition module 102C and the video signal processing module 103C of the CCU 145 execute the operations in steps S10 to S12. These operations allow the surgery system 1C to obtain the effects that are the substantially same as those obtained by the surgery system 1 according to the first embodiment.

Especially, in the surgery system 1C according to the third embodiment, in addition to the effects obtained by the surgery system 1, the single CCU 145 can generate the abdominal-cavity image and the lumen image, making it possible to downscale the surgery system 1C.

Incidentally, in the third embodiment, the determining module 104B and the control module 105B are provided in the system controller 5B such that the control module 105B is configured to control the image composition module 102C based on the determined result of the determining module 104B. The present invention is however not limited to the structure.

Specifically, installing the determining module 104B and the control module 105B in the CCU 145 permits the control module 105B to control the image composition module 102C.

In the first to third embodiments and their modifications, the lumen tube 45b joined to the second adapter 41B is joined to the adapter 43 of the manipulator 35 of the flexiblescope 31. The present invention, however, is not limited to the structure. Specifically, the lumen tube 45b can be joined to the upstream side of the gas and water supply switch 35a of the flexiblescope 31. For example, the lumen tube 45b can be joined to the light source connector 36a. This modification needs not necessarily the foot switch 44. That is, the modification allows the operator's opening and closing operation of the through hole of the switch 35a to switch between the carbon dioxide gas insufflation of the lumen BC and the interruption of the insufflation.

In the first to third embodiments and their modifications, the sub-image is superimposed on the main-image, but the present invention is not limited to the structure.

Specifically, the screen SC of the monitor 6 can be divided into, for example, two areas to display the abdominal-cavity image and the lumen image. That is, the abdominal-cavity image can be displayed on one of the areas of the screen SC, and the lumen image can be displayed on the other thereof. The displayed areas of the abdominal-cavity image and the lumen image can be switched depending on the switching of the operation mode of the gas supply apparatus 41.

In the first to third embodiments and their modifications, the rigidscope and the flexiblescope are used as observation devices for observing the inside of a patient, but the present invention is not limited to the structure. Specifically, other types of endoscopes, such as a wireless capsule endoscope or the like, or other observation devices except for endoscopes, each of which is configured to be inserted into the inside of a patient, can be used for observing the inside of the patient.

In the first to third embodiments and their modifications, the gas supply system 4 is configured to supply the predetermined gas into the abdominal cavity as the first body cavity and the lumen as the second cavity, but the present invention is not limited to the configuration. Specifically, the gas supply system 4 can be configured to supply the predetermined gas into a plurality of areas in a patient.

In the first to third embodiments and their modifications, the gas supply system 4 is configured to supply the same gas into the abdominal cavity and the lumen, but the gas supply system 4 can be configured to supply a predetermined gas into the abdominal cavity and to supply another gas into the lumen. For example, the gas supply system 4 can be configured to supply the carbon dioxide gas into the abdominal cavity and to supply air into the lumen.

Incidentally switching of the main image is not limited between the abdominal-cavity insufflation mode and the lumen insufflation mode. Specifically, it is assumed that the gas supply system 4 is configured to supply the carbon dioxide gas into a plurality of abdominal cavities, such as first and second abdominal cavities, and to supply it into a plurality of lumens, such as first and second lumens. In this assumption, the rigidscope can pickup first and second abdominal-cavity images corresponding to the first and second abdominal cavities, respectively, and the flexiblescope can pickup first and second lumen images corresponding to the first and second lumens, respectively. The gas supply apparatus can operate in first and second abdominal-cavity insufflation modes to supply the carbon dioxide gas into the first and second abdominal cavities, respectively. The gas supply apparatus can operate in first and second lumen insufflation modes to supply the carbon dioxide gas into the first and second lumens, respectively.

That is, the image processing unit can select one of the first and second abdominal-cavity images and the first and second lumen images to display it on the monitor 6 when the gas supply apparatus operates in corresponding one of the first and second abdominal-cavity insufflation modes and the first and second lumen insufflation modes.

Furthermore, it should be noted that the term “body cavity” means not only a cavity that originally exists in the body of a patient, but also a cavity (space) to be artificially formed in the body of a patient with medical instruments.

For example, the term “body cavity” according to the specification includes, as the former means, an abdominal cavity, a lumen including upper alimentary tracts (esophagus, stomach, or the like), lower alimentary tracts (large intestine, small intestine, or the like), a bladder, and a uterus.

In addition, the term “body cavity” according to the specification includes, as the later means, a cavity to secure the field of an endoscope during surgery, such as subcutaneous cavity and the like.

While there has been described what is at present considered to be the embodiment and modifications of the invention, it will be understood that various modifications which are not described yet may be made therein, and it is intended to cover in the appended claims all such modifications as fall within the true spirit and scope of the invention.

What is claimed is:

1. A medical image display system comprising:
a display for displaying a first medical image related to a first body cavity and a second medical image related to a second body cavity on a screen;
a gas supply unit configured to supply first gas into the first cavity and second gas into the second cavity; and
a switching display unit connected to the display and configured to determine whether the first gas or the second gas is supplied from the gas supply unit, the switching display unit being configured to switchably display the first medical image and the second medical image on the screen of the display based on the determined result.

2. A medical image display system according to claim 1, wherein, when the determined result represents that the first gas is supplied into the first cavity, the switching display unit is configured to:
display the first medical image on the display screen of the display; and
downsize the second medical image with respect to the first medical image to superimpose the downsized second medical image on the first medical image, and wherein, when the determined result represents that the second gas is supplied into the second cavity, the switching display unit is configured to:
display the second medical image on the display screen of the display; and
downsize the first medical image with respect to the second medical image to superimpose the downsized first medical image on the second medical image.

3. A medical image display system according to claim 1, wherein the switching display unit is configured to:
display the first medical image on the display screen of the display when the determined result represents that the first gas is supplied into the first cavity; and
display the second medical image on the display screen of the display when the determined result represents that the second gas is supplied into the second cavity.

4. A medical image display system according to claim 1, wherein the gas supply unit is configured to:
send a mode signal indicative of a first mode when supplying the first gas into the first body cavity; and
send the mode signal indicative of a second mode when supplying the second gas into the second body, and
wherein the switching display unit is configured to determine whether the mode signal sent from the gas supply unit is indicative of the first mode or the second mode to switchably display the first medical image and the second medical image on the screen of the display based on the determined result of the mode signal.

5. A medical image display system according to claim 1, further comprising a relevant information generating unit configured to generate a first relevant information image and a second relevant information image, the first relevant information image including information related to the first body cavity, the second relevant information image including information related to the second body cavity, and
wherein the switching display unit is configured to superimpose the first and second relevant information images on at least one of the first and second medical images switchably displayed on the screen of the display, the first and second relevant information images being arranged in different positions on at least one of the first and second medical images.

6. A medical image display system according to claim 1, wherein the gas supply unit comprises:
a gas supply source operative to supply predetermined gas; and
first and second delivery members configured to allow the predetermined gas supplied from the gas supply source to branch through the first delivery member and the second delivery member, respectively,
the gas supply unit being configured to supply the predetermined gas branched through the first delivery member into the first body cavity as the first gas, and to supply the predetermined gas branched through the second delivery member into the second body cavity as the second gas.

7. A medical display system according to claim 1, further comprising:
a first image-pickup apparatus configured to pick up an image inside the first body cavity as the first medical image; and
a second image-pickup apparatus configured to pick up an image inside the second body cavity as the second medical image.

8. A medical display system according to claim 7, wherein the first image-pickup apparatus is a first endoscope and the second image-pickup apparatus is a second endoscope, the first endoscope comprising:
an insertion portion to be inserted into the first body cavity;
a capturing unit configured to optically capture a first optical image inside the first body cavity; and
an image pickup unit configured to pick up the image inside the first body cavity as the first medical image based on the first optical image,
the second endoscope comprising:
an insertion portion to be inserted into the second body cavity;
a capturing unit configured to optically capture a second optical image inside the second body cavity; and
an image pickup unit configured to pick up the image inside the second body cavity as the second medical image based on the second optical image.

9. A medical image display system comprising:
means for displaying a first medical image related to a first body cavity and a second medical image related to a second body cavity on a screen;
means for supplying first gas into the first cavity and second gas into the second cavity; and
means for determining whether the first gas or the second gas is supplied from the gas supply means and for switchably displaying the first medical image and the second medical image on the screen based on the determined result.

10. A method of displaying a first medical image related to a first body cavity and a second medical image related to a second body cavity on a screen of a display, the method comprising:
supplying first gas into the first cavity and second gas into the second cavity;
determining whether the first gas or the second gas is supplied from the gas supply means; and
switchably displaying the first medical image and the second medical image on the screen of the display based on the determined result.
